(19) United States
(12) Patent Application Publication
(10) Pub. No.: US 2025/0145960 A1
(43) Pub. Date: May 8, 2025

(54) A METHOD OF DERIVATION OF MESENCHYMAL STEM CELLS FROM MAMMALIAN PLURIPOTENT STEM CELLS

(71) Applicant: The University of Hong Kong, Hong Kong (CN)

(72) Inventors: Zhongjun Zhou, Hong Kong (CN); Wei Jin, Hong Kong (CN)

(21) Appl. No.: 18/837,834

(22) PCT Filed: Jan. 30, 2023
(86) PCT No.: PCT/CN2023/073818

§ 371 Date: Aug. 12, 2024

Related U.S. Application Data
(60) Provisional application No. 63/267,811, filed on Feb. 10, 2022.

(51) Int. Cl. C12N 5/0775

(52) U.S. Cl. CPC C12N 5/0668; C12N 2501/15; C12N 2501/415; C12N 2506/45

(57) ABSTRACT

The subject invention pertains to methods of derivation of MSCs from both human or other mammal iPSCs and embryonic stem cells (ESCs) via a temporal induction of neural ectoderm in chemically defined media. In certain embodiments, the methods use a two-stage culture.

### BACKGROUND OF THE INVENTION

Mesenchymal stem cells (MSCs) are multipotent progenitor cells firstly isolated from bone marrow.¹ MSCs are characterized by their fibroblast-like morphology, high potential of osteogenesis, adipogenesis and chondrogenesis, and positive for a panel of typical surface markers, including CD73, CD90 and CD105.² Because of the low immunogenicity, high regenerative capability and strong immunomodulatory properties, both allogenic and autologous MSCs have been deployed in a large number of phase I-III clinical trials for degenerative diseases and autoimmune disorders, such as ischemic heart diseases, neurodegenerative disorders, graft versus host disease and Crohn's disease, as well as hematopoietic stem cell (HSC) engraftment failure.^{3,4} Both ex vivo and animal studies have shown that MSCs can home to sites of the injuries or inflammation to create a favorable microenvironment to support the survival and functional recovery of injured cells, promoting their proliferation via direct cell-cell contact or paracrine secretion.⁵ In addition, MSCs in culture secrete a myriad of bioactive molecules, including growth factors, cytokines, chemokines, and mRNA/microRNA-containing macrovesicles,^{6, 7} which have been successfully used in clinical cosmetology.⁸ Therefore, MSCs have emerged as the most frequently used bio-reagent in the fields of regenerative medicine and tissue engineering.⁹

MSCs have been successfully isolated and expanded from multiple human tissues including bone marrow, adipose tissue, umbilical cord, muscle, liver, cartilage, and lung.^{10, 11} However, there are several drawbacks in MSCs derived from adult tissues. The quantity of MSCs isolated from primary tissues is far from sufficient for clinical applications¹² and the primary MSCs usually exhibit limited proliferative capacity, altered functionality and compromised differentiation potential over long-term culture.¹³ In addition, tissue-derived MSCs suffer from tissue and donor variability, especially with the regard of age and disease-status of the donors,^{14, 15} which may explain the discrepancy in the efficacies of preclinical evaluation of MSCs. On the other hand, although MSCs exhibit low immunogenicity in general, the allogenic MSCs from bone marrow (BM-MSCs) were reported to induce undesired immune response in several animal models.¹⁶⁻¹⁸

Human pluripotent stem cells (hPSCs), including embryonic stem cells and induced pluripotent stem cells, possess the capacity of indefinite proliferation and differentiation into all three germ layers, making hPSCs an ideal, easily accessible and safe source for large-scale production of high-quality MSCs. Up to now, various strategies and modified protocols have been developed to generate MSCs from hPSCs. Barberi et al. co-cultured human embryonic stem cells (hESCs) with mouse OP9 cells for 40 days followed by FACS sorting for CD73+ cells to enrich the MSC population.¹⁹ MSCs can be derived and purified with typical surfaces markers from the spontaneous differentiation of embryonic body (EB), a sphere culture derivative of hPSCs. The EB-mediated derivation of MSCs has been extensively optimized to improve the yields, for example, plating EB to gelatin coated-dishes and expanding monolayer cells before sorting, or continually splitting the cells until a homogeneous fibroblastic morphology appears. Lian and colleagues cultured hESCs directly for 7 days in MSCs medium supplemented with PDGF-AB and bFGF without EB formation followed by sorting for CD105⁺/CD24⁻ cells.²⁰ These methods are time-consuming and inefficient. In addition, the use of animal components makes the MSC production non-compliant with the clinical requirements. To establish a direct induction platform, Sánchez. et al. blocked the TGF-beta signaling with small molecule inhibitor SB431542. This could promote the induction of MSCs from hESCs, but not induced pluripotent stem cells (iPSCs).²¹ Based on this method, Zhao et al. prolonged the induction in the presence of SB431542 and 7.5% CO₂ to enable MSCs derivation from iPSCs.²² Later, Wang et al. generated MSCs via transforming hESCs into a trophoblast-like stage before differentiating to MSCs,²³ which may give rise to ethical controversies in clinical applications of hESCs. Neural crest cells (NCCs) belong to a transitional multi-potent population emerged during development of embryonic ectoderm in vertebrates, which can be easily directed towards mesenchymal derivatives.²⁴ Fukuta et al. previously described the 7-days induction of hPSCs to neural ectoderm with TGF-beta inhibition and WNT activation followed by FACS sorting for p75^{high} population of NCCs before MSCs induction.²⁵ However, the efficiency of NCC production is relatively low and FACS sorting is not practical for large-scale production of MSCs. In addition, highly differentiated pure NCCs requires specific medium with sophisticated composition and approximately 20 days induction, adding extra burden for MSCs production.²⁶

MSCs are emerging as the mainstay of regenerative medicine because of their ability to differentiate into multiple cell lineages. The infinite proliferative potential of human pluripotent stem cells (PSCs) grants an unlimited supply of MSCs. Despite their great potential in therapeutic applications, several drawbacks have hindered its clinical translation, including limited number of replications, compromised potential, and altered function in late passages. Therefore, there remains a need for an efficient method for the production of MSCs from pluripotent stem cells for potential clinical application.

### BRIEF SUMMARY OF THE INVENTION

The subject invention pertains to methods of derivation of MSCs from both human or other mammal iPSCs and embryonic stem cells (ESCs) via a temporal induction of neural ectoderm in chemically defined media. In certain embodiments, the methods use a two-stage culture. In certain embodiments, the pluripotent stem cells can be cultured in a first medium comprising a cell basal medium, a cell culture supplement, a non-essential amino acids solution, sodium pyruvate, insulin, transferrin, selenium, L-ascorbic acid, an antibiotic solution, a GSK-3 inhibitor, and an inhibitor of activin receptor-like kinase receptors. In certain embodiments, the resulting MSCs can be cultured in a second medium containing a basal medium, FBS, a cell culture supplement, bFGF, TGFB1, and an antibiotic solution. The pluripotent stem cells can be culture in the first medium for about 1 to about 8 days, preferably about 2 to about 6 days, or most preferably about 4 days. The MSCs can be culture in the second medium for about 1 to about 14 days, preferably about 2 to about 12 days, most preferably about 4 to about 10 days.

### BRIEF DESCRIPTION OF THE DRAWINGS

FIGS. 1A-1F. Generation and characterization of human iPSCs with an integration-free minicircle vector. FIG. 1A. Morphology of human primary dermal fibroblasts (1), iPSCs generated from fibroblasts cultured on feeder layer (2) and feeder-free plate (3). Scar bar 100 μm. FIG. 1B. Expression of pluripotency-related genes OCT3/4, NANOG, SOX2, REX1, GDF3, hTERT and DNMT3b, analyzed by qPCR. Gene expression was normalized to endogenous GAPDH and was plotted against fibroblast, H9 served as a positive control. Data represent mean±S.D. n=3. FIG. 1C. DNA methylation detected by Bisulfite sequencing in the genomic region of OCT3/4 promoter in iPSCs and its parental dermal fibroblasts. Open and filled circles represents unmethylated and methylated CpGs, respectively. Each row represents bisulfite sequencing result from a given amplicon and ten amplicons were analyzed. FIG. 1D. Immunostaining of pluripotency markers SSEA-4, TRA60-1, NANOG and OCT3/4 (Scar bar 10 μm), Alkaline Phosphatase (AP) staining and embryoid body (EB) formation of iPSCs. Scar bar 100 μm. FIG. 1E. Karyotyping assay of iPSC with a normal diploid female karyotype. FIG. 1F. Teratoma formation of iPSC's in the testis of NOD/SCID mice. Teratoma and H&E staining of paraffin sections to reveal three germ layers. Scar bar 100 μm.

FIGS. 2A-2L. Generation of MSCs from pluripotent stem cells by two-step induction via neutralized ectoderm as a differentiation intermediate. FIG. 2A. Schematic illustration of two-step procedure for MSCs induction. Human pluripotent stem cells are treated with SMAD inhibitor and WNT activator for 5-7 days to differentiate to the neural ectoderm. Cells are then split before further incubated with medium supplemented with bFGF/EGF. Fibroblast-like cell morphology were observed around day 4-10. Cells were expanded for another 2-3 passages followed by FACS analysis of typical MSC surface markers. Lower panel shows the morphology of initial human iPSCs, intermediate neural ectoderm and fibroblast-like MSCs. Scar bar 100 μm. FIGS. 2B-2E. Expression of neural ectoderm genes (SOX10, SOX2, SOX9, FOXD3, p75, PAX3, ZIC, and AP2a) and pluripotency related genes (OCT3/4, NANOG, and REX1) examined by qPCR. Gene expression was normalized to endogenous GAPDH. Relative mRNA levels were plotted against that in iPSCs. Data represent mean±S.D. n=3; *p<0.05, **p<0.01, ***p<0.001, ****p<0.0001, ns, not significant; one-way ANOVA coupled with Tukey's post hoc test was used for statistical analysis. FIGS. 2F-2J. Flow cytometry analyses of typical positive (CD73, CD90, CD105 and CD44) and negative (CD45, CD34, CD11b, CD19 and HDL-DR) MSCs surface markers. FIG. 2K. MSCs are induced toward adipogenesis, chondrogenesis and osteogenesis in vitro for three weeks. Adipocyte, chondrocyte, and osteoblast are detected by staining of Oil red O, Alizarin red S and Alcian blue. Scar bar 100 μm. FIG. 2L. Colony formation of MSCs. MSCs are stained with crystal violet. A representative colony in high magnification is shown on the right.

FIGS. 3A-3Q. Comparison of iPSCs- and ESCs-derived MSCs with primary bone marrow MSCs. FIGS. 3A-30. Flow cytometry analyses of typical positive (CD73, CD90, CD105 and CD44) and negative (CD45, CD34, CD11b, CD19 and HDL-DR) surface markers for MSCs in WT-MSCs, H1-MSCs and BM-MSCs. FIG. 3P. Adipogenesis, chondrogenesis and osteogenesis of WT-MSCs, H1-MSCs and BM-MSCs. Adipocyte, chondrocyte, and osteoblast are detected by the staining of Oil red O, Alizarin red S and Alcian blue, respectively. Scar bar 100 μm. FIG. 3Q. Correlation co-efficient (R2) of gene expression profiling between WT-MSCs, H1-MSCs and BM-MSCs.

FIGS. 4A-4I. Derivation and characterization of HGPS-MSCs. FIG. 4A. Immunostaining of SSEA-4, TRA60-1 and NANOG and OCT3/4 (Scar bar 10 μm), alkaline Phosphatase (AP) activity and embryoid body (EB) formation of HGPS-iPSCs. Scar bar 100 μm. FIG. 4B. DNA methylation in the genomic region at OCT3/4 promoter in HGPS-iPSCs and its parental dermal fibroblasts. Open and filled circles represent unmethylated and methylated CpGs, respectively.

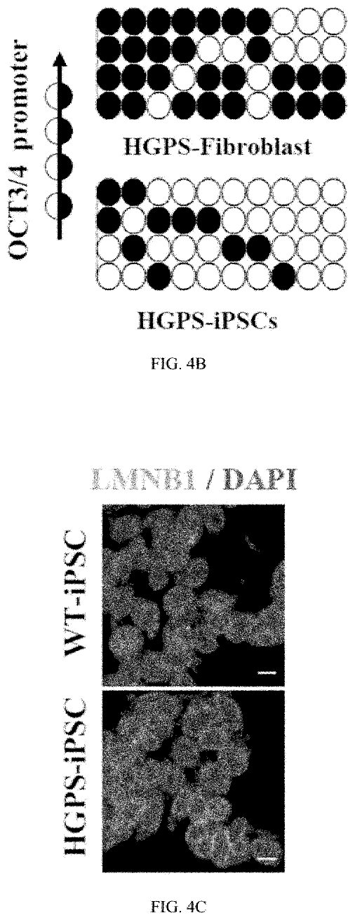

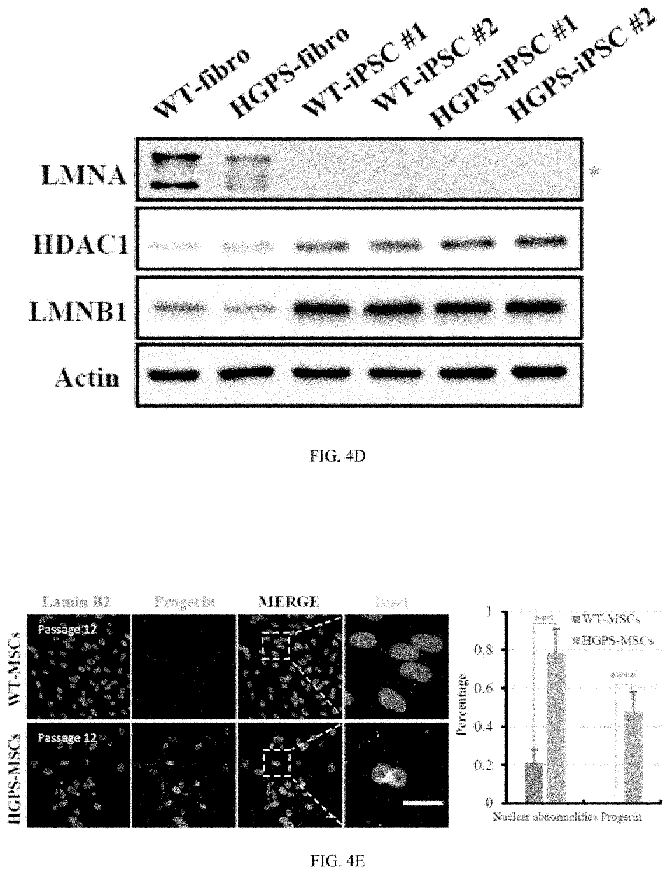

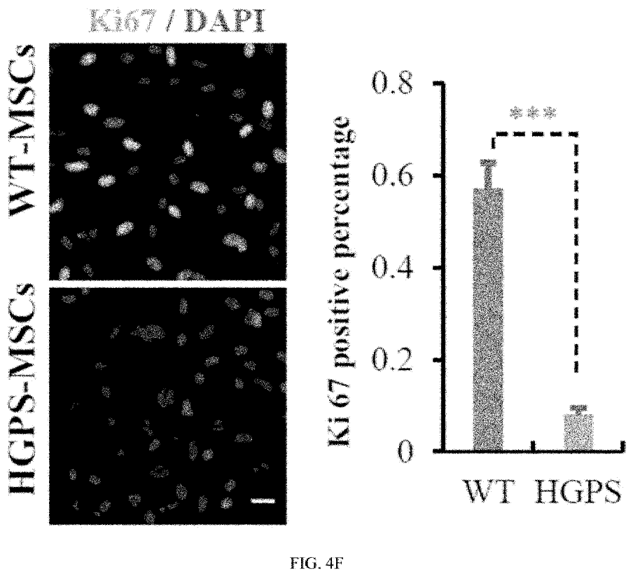

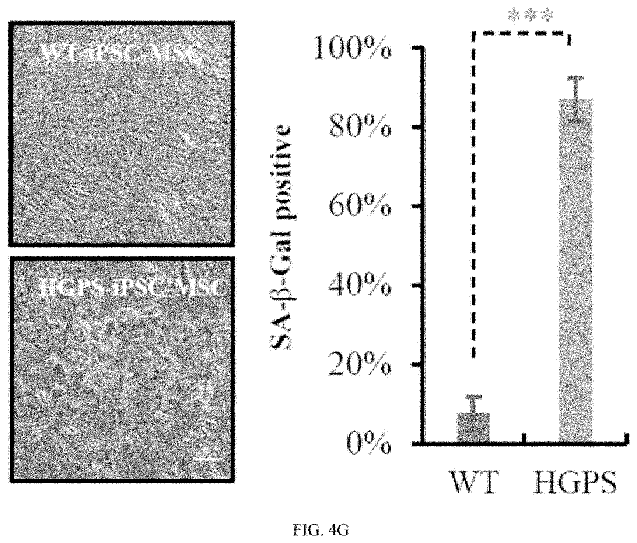

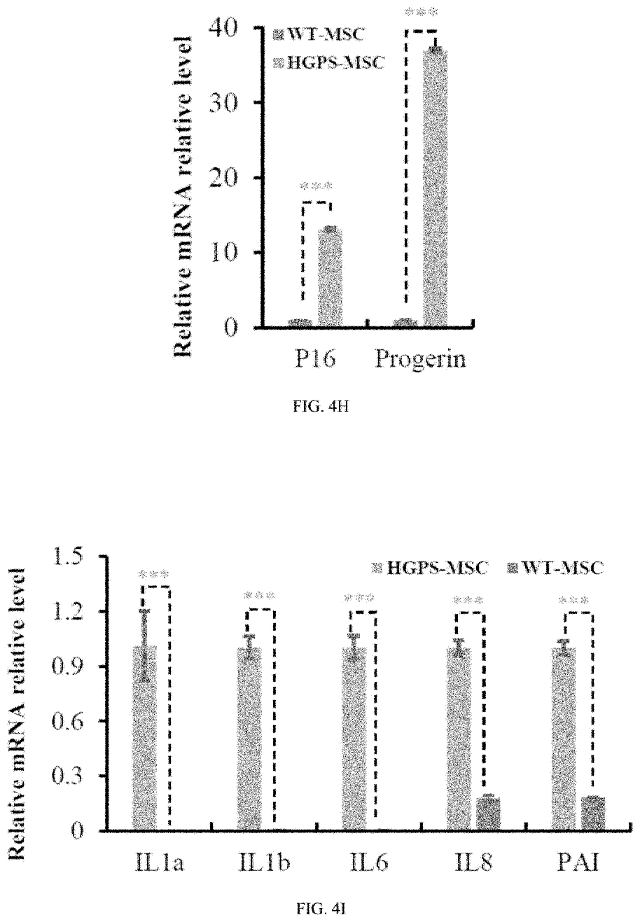

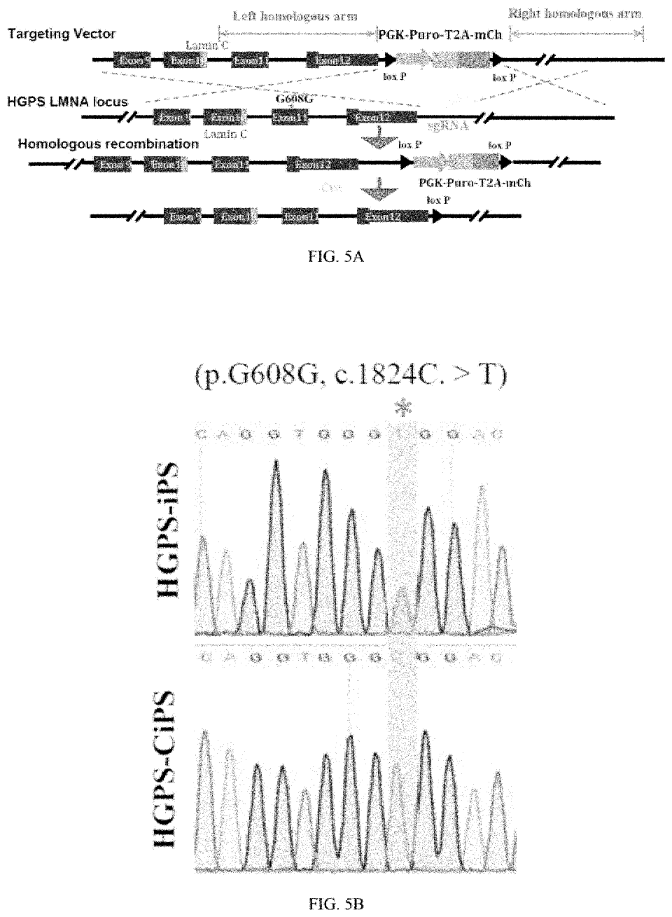

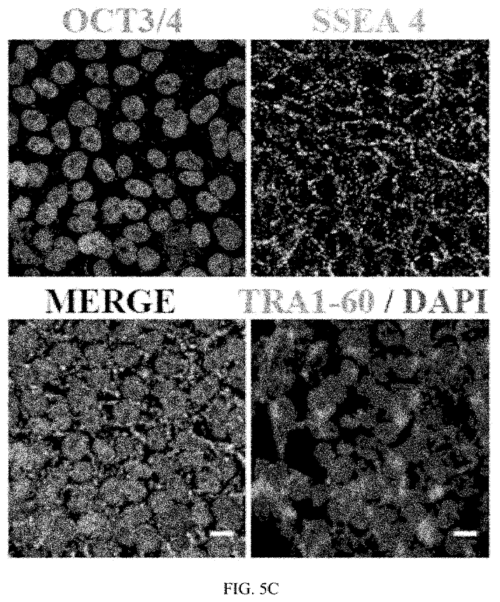

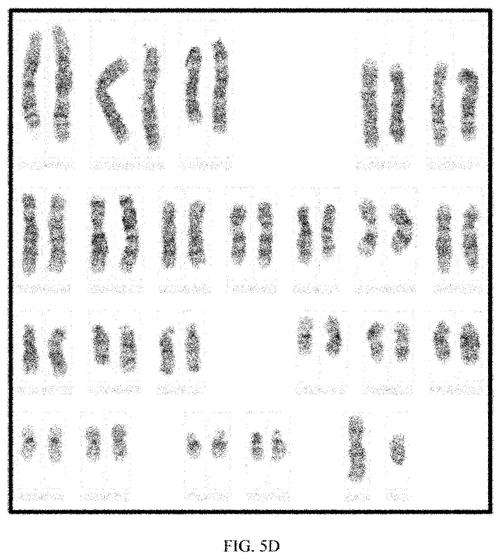

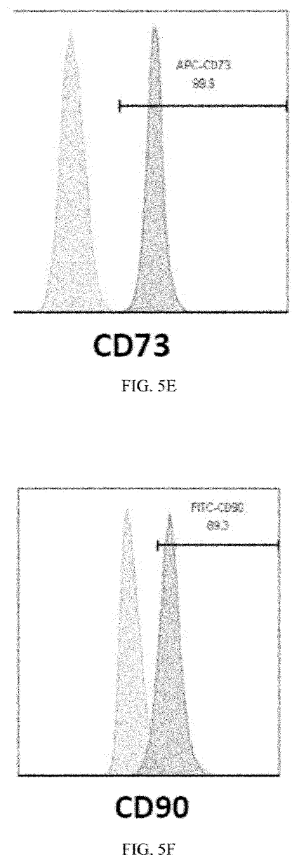

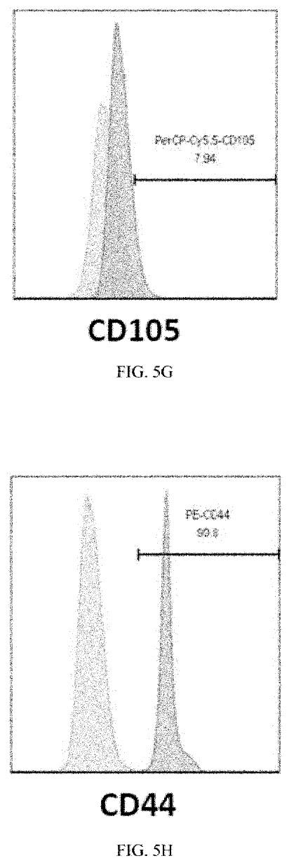

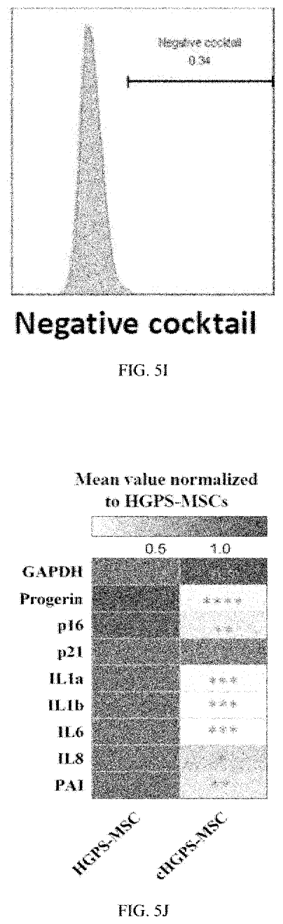

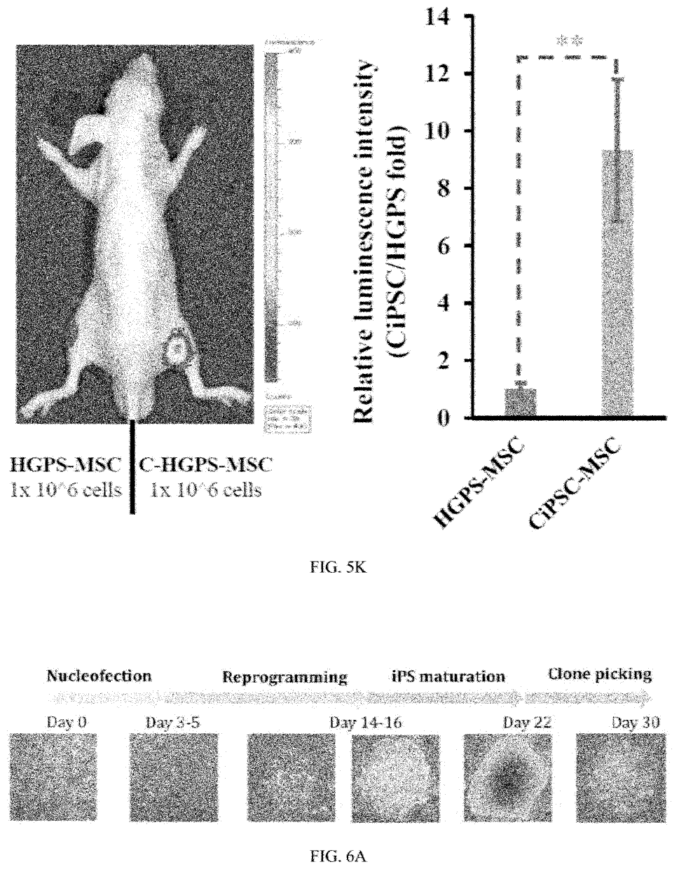

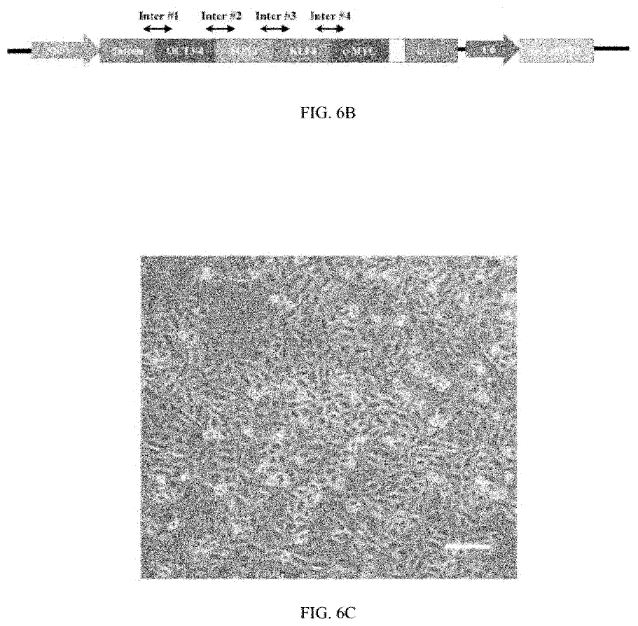

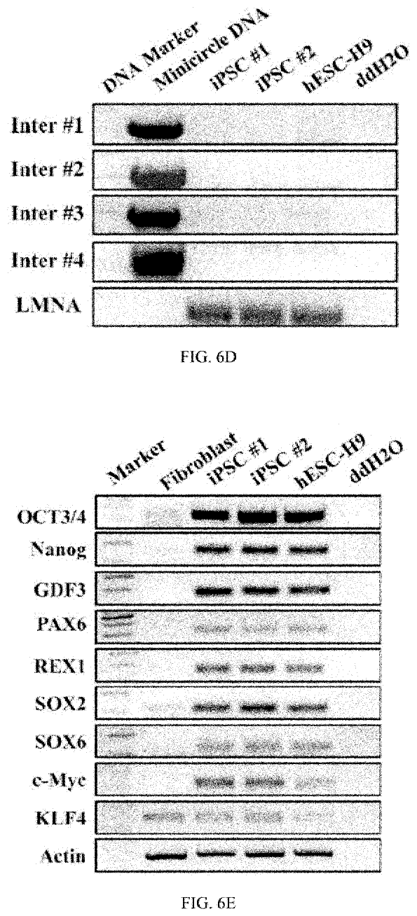

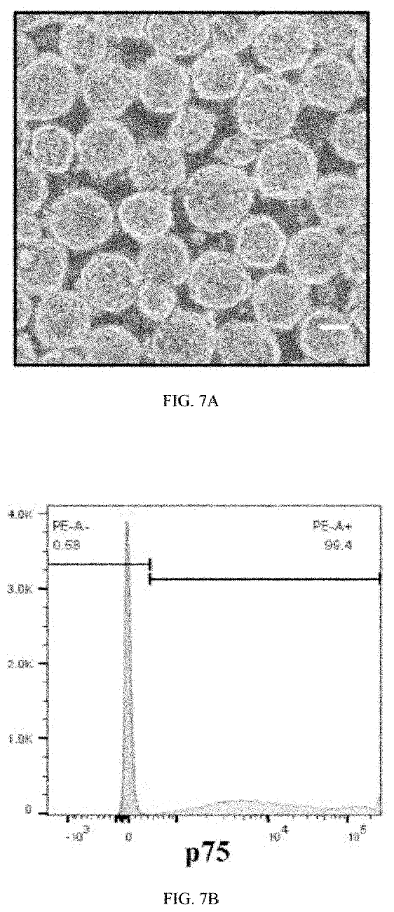

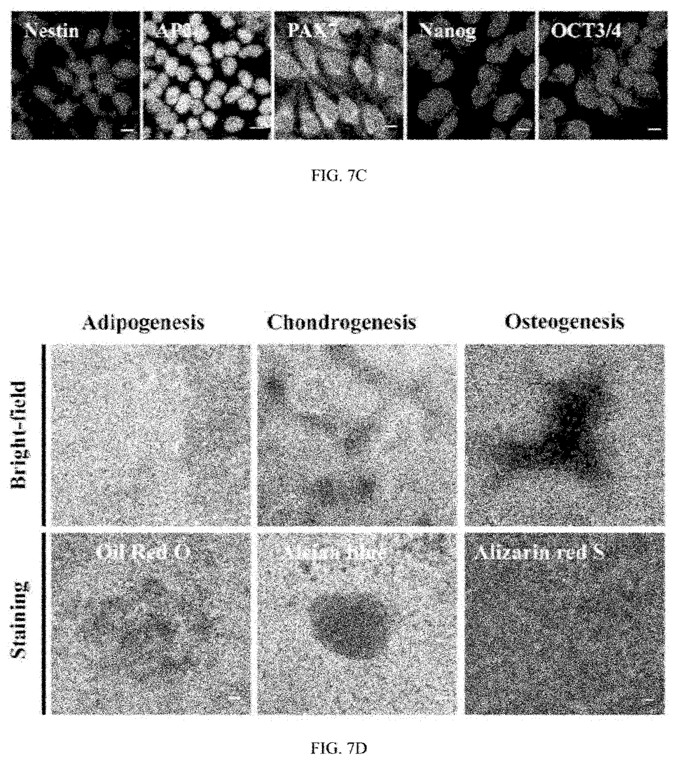

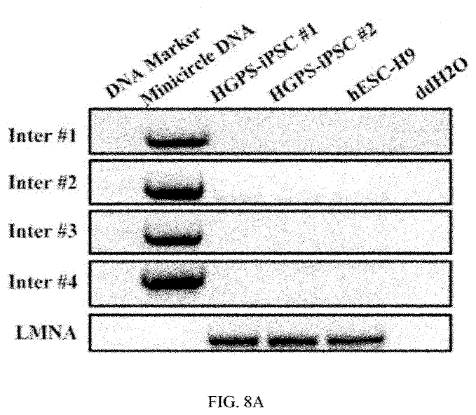

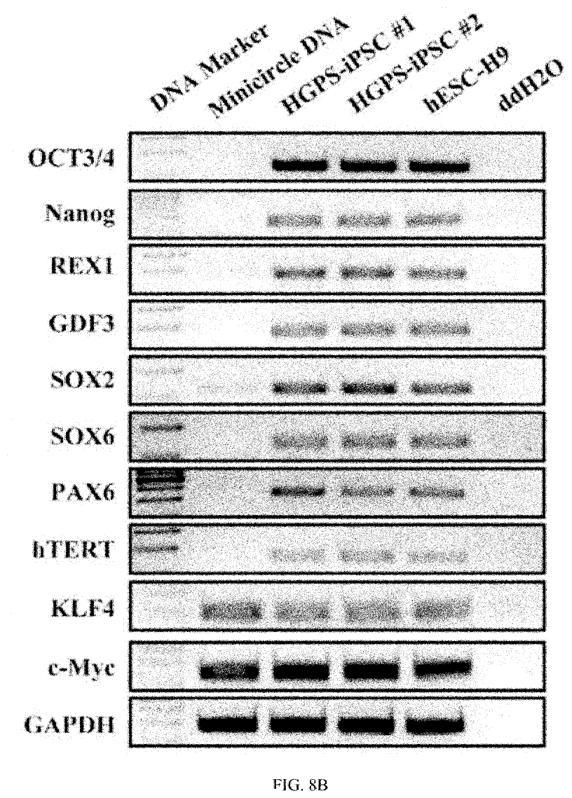

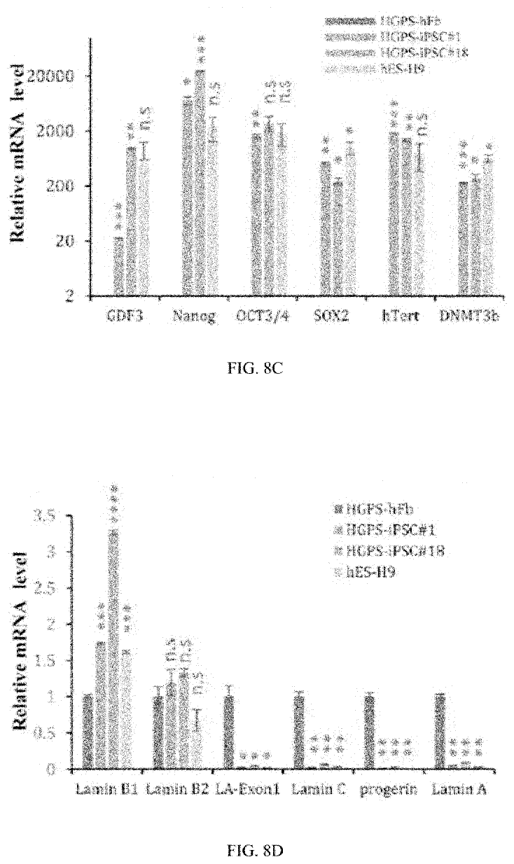

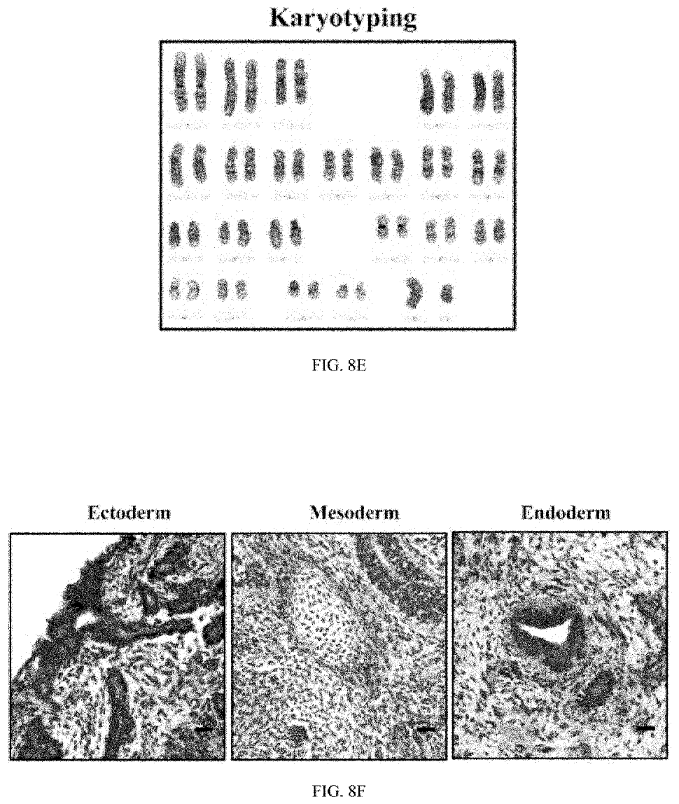

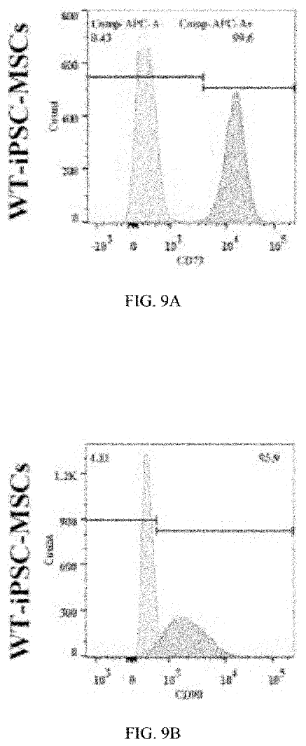

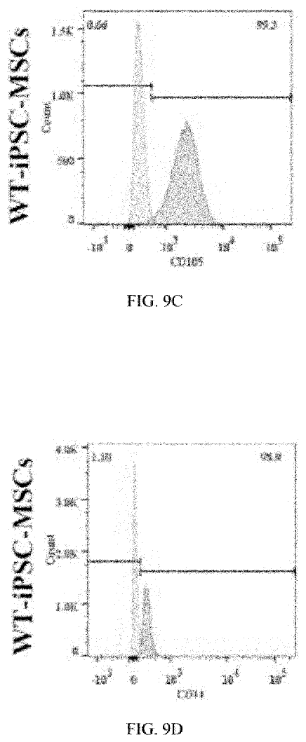

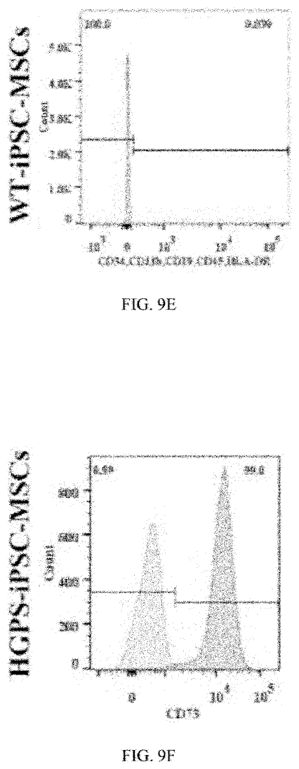

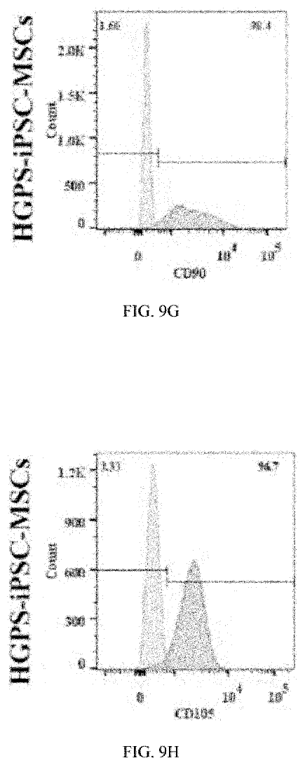

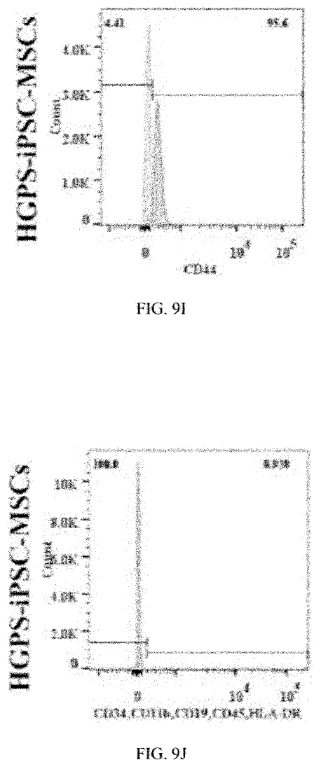

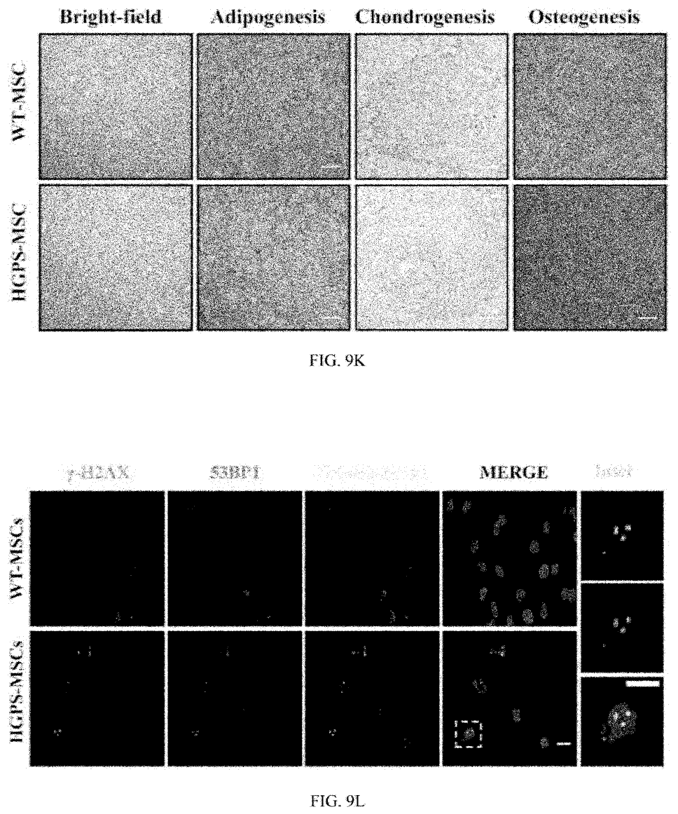

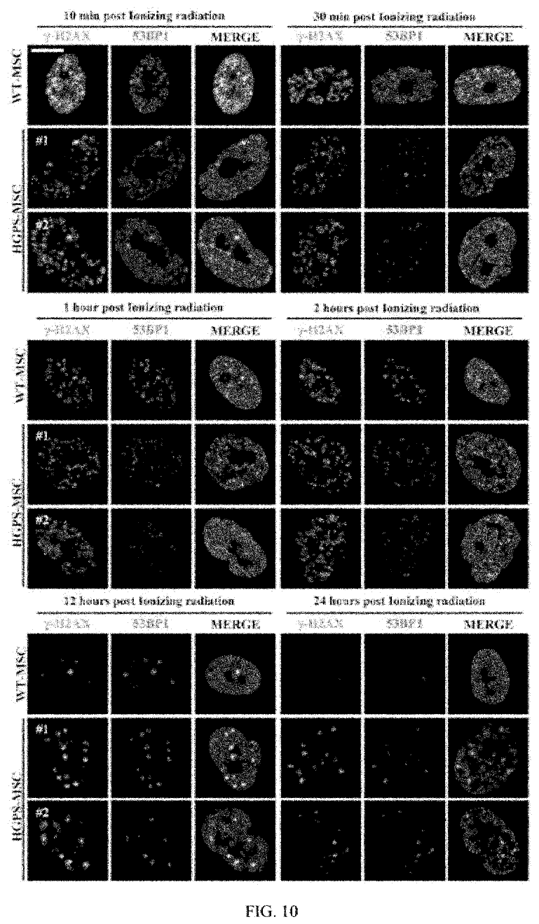

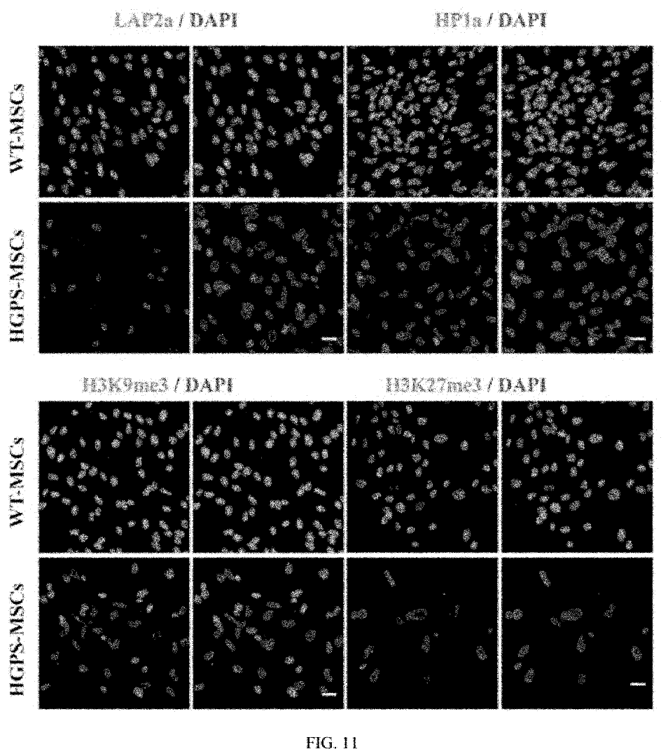

Each row represents bisulfite sequencing result from a given amplicon and ten amplicons were analyzed. FIG. 4C. Immunostaining of lamin B1 in HGPS-iPSCs and WT-iPSCs. Scar bar 10 μm. FIG. 4D. Expression of nuclear lamins and HDAC1 examined by Western blotting. β-Actin serves as a loading control. FIG. 4E. Co-immunostaining of lamin B2 and progerin in HGPS-iPSCs derived MSCs, scar bar 10 μm. Percentages of cells exhibiting nuclear deformation and progerin expression were analyzed. Data represent mean±S.E.; n=4; *P<0.05, **P<0.01, ***P<0.005, ****P<0.001*p<0.05, **p<0.01, ***p<0.001, ****p<0.0001; ns, not significant; unpaired two-tailed Student's t-test is used for statistical analyses. FIG. 4F. Immunoblotting of Ki 67 in HGPS-iPSCs and WT-iPSCs derived MSCs. Scar bar 100 μm. Data represent mean±S.E.; n=5; *P<0.05, **P<0.01, ***P<0.005, ****P<0.001; ns, not significant; unpaired two-tailed Student's t-test is used for statistical analyses. FIG. 4G. SA-β-gal 20 staining of MSCs derived from HGPS-iPSCs and WT-iPSCs. Cellular senescence was quantified by SA-β-gal positive cell number. Data represent mean±S.E.; n=5, *p<0.05, **p<0.01, *** p<0.001, ****p<0.0001, ns, not significant; one-way ANOVA coupled with Tukey's post hoc test is used for statistical analyses. FIGS. 4H-4I. Transcription of p16, p21 and SASP-related inflammatory cytokine examined by qPCR. Data represent mean±S.D.; n=4; *p<0.05, ** p<0.01, ***p<0.001, ****p<0.0001, ns, not significant; one-way ANOVA coupled with Tukey's post hoc test is employed for statistical analyses.

FIG. 5A-5K. Rescue of premature senescence in mutation-rectified HGPS-MSCs. FIG. 5A. Schematic illustration of the strategy to rectify LMNA mutation in HGPS-iPSCs by CRISPR/Cas9-mediated gene targeting. The small guide RNA targets the downstream of 3′UTR of LMNA gene with two homologous arms adjacent to sgRNA covering 1.5 kb upstream and downstream of genomic DNA, respectively. The double selective markers (puromycin resistant gene and fluorescent protein mCherry) are flanked by the homologous arms. The mutation corrected clones were screened and selected and confirmed by Sanger sequencing. Selection markers and other foreign DNA sequences were removed by transient expression of Flpase.

FIG. 5B. Sanger sequencing to confirm mutation rectification of LMNA gene in a HGPS-CiPSCs clone, in comparison to its parental HGPS-iPSCs. FIG. 5C. Immunostaining of pluripotency markers SSEA-4, TRA60-1, NANOG and OCT3/4 (Scar bar 10 μm) in HGPS-CiPSCs. Scale bar, 10 μm. FIG. 5D. Karyotyping of HGPS-CiPSCs showing normal diploid male karyotype. FIGS. 5E-5I. Flow cytometry analyses of surface markers in MSCs generated from HGPS-CiPSCs. Note that more than 95% of the cells are positive for CD73, CD90, CD105 and CD44, and negative for CD45, CD34, CD11b, CD19 or HDL. FIG. 5J. Heatmap of gene expressions of progerin, p16, p21 and SASP-related genes (IL1a, IL1b, IL6, IL8 and PAI), based on mean value in the MSCs derived from HGPS-iPSCs and HGPS-CiPSCs. Data represent mean±S.D.; n=4; * p<0.05, **p<0.01, ***p<0.001, ****p<0.0001, ns, not significant; one-way ANOVA coupled with Tukey's post hoc test is employed for statistical analyses. FIG. 5K. Comparison of the In vivo bioluminescence between MSCs derived from mutation rectified HGPS-CiPSCs and MSCs derived from HGPS-iPSCs. Bioluminescent intensities were quantified according to the luciferase activities. Data represent mean±S.D.; n=3; **p<0.01; one-way ANOVA coupled with Tukey's post hoc test is employed for statistical analyses.

FIGS. 6A-6E. Generation and characterization of human iPSCs with an integration-free minicircle vector. FIG. 6A. Schematic illustration of iPSCs generation timeline and cellular morphology changes along reprogramming. FIG. 6B. Diagram of four-in-one minicircle vector used for non-integrating reprogramming. Yamanaka factors (OCT3/4, KLF4, c-Myc, SOX2) and fluorescent protein mCherry are assembled within one gene cassette, linked with 2A peptide under SSFV promoter; U6 promoter-driven TP53 shRNA used to increase the efficiency of iPSCs generation. FIG. 6C. Human urinary primary epithelial cells expanded for 10 days. Scar bar 100 μm. FIG. 6D. PCR to examine the potential insertion of the reprogramming construction in 4 iPSC clones. Four pairs of PCR primer set to amply Inter #1, Inter #2, Inter #3, and Inter #4 regions are shown in panel a. Genome LMNA locus serves as endogenous control. Minicircle DNA serves as positive control whereas human embryonic stem cell H9 serves as negative control. FIG. 6E. Reverse transcription PCR (RT-PCR) to detect the expression of pluripotency genes including OCT3/4, NANOG, SOX2, SOX6, Klf4, REX1, GDF3, c-Myc, PAX 6 in iPSC and parental fibroblasts. Actin serves as a control.

FIGS. 7A-7D. Generation of MSCs from pluripotent stem cells by a simple two-step induction via neutralized ectoderm as a differentiation intermediate. FIG. 7A. Sphere formation of neural ectoderm cells after 5 days induction of human iPSCs. Scar bar 100 μm.

FIG. 7B. Flow cytometry analysis of the neural ectoderm intermediate, showing the vast majority of cells are p75 positive. FIG. 7C. Immunostaining of the neural ectoderm intermediate with HNK1, AP2a, PAX7, Nestin, NANOG and OCT3/4. Scar bar 10 μm. FIG. 7D. Differentiation of neural ectoderm intermediate toward adipogenesis, chondrogenesis and osteogenesis. Adipocyte, chondrocyte, and osteoblast are detected by the staining of Oil red O, Alizarin red S and Alcian blue, respectively. Scar bar 100 μm.

FIGS. 8A-8F. Characterization of HGPS patient specific iPSCs. FIG. 8A. PCR to examine potential insertion of reprogramming construct in two HGPS-iPSCs clones using 4 pairs of primers to amplify the designated region indicated in FIG. 6A. Genome LMNA locus serves as endogenous control. Minicircle DNA serves as positive control and genomic DNA from human embryonic stem cells H9 serves as negative control. FIG. 8B. Reverse transcription PCR (RT-PCR) to detect pluripotency related genes including OCT3/4, Nanog, SOX2, SOX6, Klf4, REX1, GDF3, c-Myc, and PAX 6 in two HGPS-iPSCs clones and parental fibroblasts. Actin serves as a control. FIG. 8C. qPCR analyses of pluripotency related genes OCT3/4, NANOG, SOX2, REX1, GDF3, hTERT and DNMT3b in two HGPS-iPSCs clones. Gene expression was normalized to the endogenous GAPDH. Relative mRNA levels were plotted against to that in parental fibroblasts. H9 ESCs serve as the positive control. Data represent mean±S.D.; n=3. FIG. 8D. qPCR analyses of nuclear lamins including lamin B1, lamin B2, lamin A, lamin C and progerin in HGPS-iPSCs. Gene expression was normalized to the endogenous GAPDH. Relative mRNA levels were plotted against to that in parental fibroblasts. Human ESCs H9 serve as a positive control. Data represent mean±S.D.; n-3. FIG. 8E. Karyotyping of HGPS-iPSCs showing normal diploid female karyotype. FIG. 8F. Teratoma formation of HGPS-iPSCs in the testis of NOD/SCID mice. H&E staining of teratoma paraffin sections to reveal three germ layers. Scar bar 100 μm.

FIGS. 9A-9L. Generation and characterization of HGPS iPSCs derived MSCs. FIGS. 9A-9J. Flow cytometry analyses of typical positive (CD73, CD90, CD105 and CD44) and negative (CD45, CD34, CD11b, CD19 nor HDL-DR) MSCs surface markers in WT-MSCs and HGPS-MSCs. FIG. 9K. Morphology and multiple differentiation potential of WT-MSCs and HGPS-MSCs. Adipocytes, chondrocytes, and osteoblasts detected by staining of Oil red O, Alizarin red S and Alcian blue, respectively. Scar bar 100 μm. FIG. 9L. Co-immunostaining of γ-H2AX and 53BP1 in WT-MSCs and HGPS-MSCs at passage 15. The white box in HGPS-MSCs is enlarged in the right panels. Scar bar 10 μm.

FIG. 10. The kinetics of DNA damage checkpoint response in WT-MSCs and HGPS-MSCs. Immunofluorescence analyses of γ-H2AX and 53BP1 in WT-MSCs and HGPS-MSCs at different time points upon 10 Gy of γ-irradiation. Scale bars, 10 μm.

FIG. 11. Epigenetic alternations in HGPS MSCs. Immunofluorescence analyses of LAP2a, HP1a, H3K9me3 and H3K27me3 in WT-MSCs and HGPS-MSCs. Scale bars, 10 μm.

### BRIEF DESCRIPTION OF THE SEQUENCES

SEQ ID NO: 1: MIP247-Inter-F1 primer

SEQ ID NO: 2: MIP247-Inter-R1 primer

SEQ ID NO: 3: MIP247-Inter-F2 primer

SEQ ID NO: 4: MIP247-Inter-R2 primer

SEQ ID NO: 5: MIP247-Inter-F3 primer

SEQ ID NO: 6: MIP247-Inter-R3 primer

SEQ ID NO: 7: MIP247-Inter-F4 primer

SEQ ID NO: 8: MIP247-Inter-R4 primer

SEQ ID NO: 9: HGPS-GT-F1 primer

SEQ ID NO: 10: pG13.0-sgRNA-R primer

SEQ ID NO: 11: Sox2-qRT-F primer

SEQ ID NO: 12: Sox2-qRT-R primer

SEQ ID NO: 13: Nanog-qRT-F primer

SEQ ID NO: 14: Nanog-qRT-R primer

SEQ ID NO: 15: Rex1-qRT-F primer

SEQ ID NO: 16: Rex1-qRT-R primer

SEQ ID NO: 17: Klf4-qRT-F primer

SEQ ID NO: 18: Klf4-qRT-R primer

SEQ ID NO: 19: Myc-qRT-F primer

SEQ ID NO: 20: Myc-qRT-R primer

SEQ ID NO: 21: DNMT3B-qRT-F primer

SEQ ID NO: 22: DNMT3B-qRT-R primer

SEQ ID NO: 23: TERT-qRT-F primer

SEQ ID NO: 24: TERT-qRT-R primer

SEQ ID NO: 25: GDF3-qRT-F primer

SEQ ID NO: 26: GDF3-qRT-R1 primer

SEQ ID NO: 27: 18 S-rRNA-qRT-F primer

SEQ ID NO: 28: 18 S-rRNA-qRT-R primer

SEQ ID NO: 29: GAPDH-qRT-F primer

SEQ ID NO: 30: GAPDH-qRT-R primer

SEQ ID NO: 31: LaminB1-qRT-F primer

SEQ ID NO: 32: LaminB1-qRT-R primer

SEQ ID NO: 33: LaminB2-qRT-F primer

SEQ ID NO: 34: LaminB2-qRT-R primer

SEQ ID NO: 35: LC-qRT-F primer

SEQ ID NO: 36: LC-qRT-R primer

SEQ ID NO: 37: Progerin-qRT-F primer

SEQ ID NO: 38: Progerin-qRT-R primer

SEQ ID NO: 39: hLA-Exon1-qRT-F1 primer

SEQ ID NO: 40: LA-Exon1-qRT-R1 primer

SEQ ID NO: 41: LaminA-qRT-F1 primer

SEQ ID NO: 42: LaminA-qRT-R1 primer

SEQ ID NO: 43: OCT4-BS-F1 primer

SEQ ID NO: 44: OCT4-BS-R1 primer

SEQ ID NO: 45: AP2a-qRT-F1 primer

SEQ ID NO: 46: AP2a-qRT-R1 primer

SEQ ID NO: 47: HNK1-qRT-F1 primer

SEQ ID NO: 48: HNK1-qRT-R1 primer

SEQ ID NO: 49: p75-qRT-F1 primer

SEQ ID NO: 50: p75-qRT-R1 primer

SEQ ID NO: 51: PAX3-qRT-F1 primer

SEQ ID NO: 52: PAX3-qRT-R1 primer

SEQ ID NO: 53: SOX10-qRT-F1 primer

SEQ ID NO: 54: SOX10-qRT-R1 primer

SEQ ID NO: 55: ZIC1-qRT-F1 primer

SEQ ID NO: 56: ZIC1-qRT-R1 primer

SEQ ID NO: 57: SOX9-qRT-F1 primer

SEQ ID NO: 58: SOX9-qRT-R1 primer

SEQ ID NO: 59: p16-qRT-F1 primer

SEQ ID NO: 60: p16-qRT-R1 primer

SEQ ID NO: 61: p21-qRT-F1 primer

SEQ ID NO: 62: p21-qRT-R1 primer

SEQ ID NO: 63: IL1α-qRT-F1 primer

SEQ ID NO: 64: IL1α-qRT-R1 primer

SEQ ID NO: 65: IL1β-qRT-F1 primer

SEQ ID NO: 66: IL1β-qRT-R1 primer

SEQ ID NO: 67: IL6-qRT-F1 primer

SEQ ID NO: 68: IL6-qRT-R1 primer

SEQ ID NO: 69: IL8-qRT-F1 primer

SEQ ID NO: 70: IL8-qRT-R1 primer

SEQ ID NO: 71: PAI-1-qRT-F1 primer

SEQ ID NO: 72: PAI-1-qRT-R1 primer

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” is used provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, the “stem cell” refers to an undifferentiated or partially differentiated cell capable of self-renewal and differentiation/proliferation. Subpopulations of stem cells include, for example, pluripotent stem cell, multipotent stem cell, unipotent stem cell and the like, according to the differentiation potency.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims.

Methods of Producing Mesenchymal Stem Cells from Pluripotent Stem Cells

In certain embodiments, mesenchymal stem cells can be produced from pluripotent stem cells or embryonic stem cells. In certain embodiments, pluripotent stem cells are cells that are capable of self-renewing by dividing and developing into the three primary germ cell layers of the early embryo and therefore producing any cell or tissue of the body but not extra-embryonic tissues such as, for example, the placenta. In certain embodiments, multipotent stem cells are cells that are capable of self-renewal by dividing and to developing into multiple specialized cell types found in a specific tissue or organ. In certain embodiments, unipotent stem cells are cells that can produce only one cell type but have the capability of self-renewal that distinguishes them from non-stem cells, examples of which include germ line stem cell (producing sperm) and an epidermal stem cell (producing skin).

In certain embodiments, embryonic stem cells (ESCs), embryonic germ cells (EGCs), and induced pluripotent stem (iPSC) cells are examples of pluripotent stem cells. Most adult stem cells are multipotent stem cells including somatic stem cells such as mesenchymal stem cell, hematopoietic stem cell, neural stem cell, myeloid stem cell and germ line stem cell, and the like. The multipotent stem cell is preferably a mesenchymal stem cell which broadly means a population of stem cells or progenitor cells thereof, which can differentiate into all or some of the mesenchymal cells, such as osteoblast, chondroblast, lipoblast and the like.

In certain embodiments, a pluripotent stem cell, such as, for example, a human pluripotent stem cells (hPSC) can be cultured in a feeder-free media using, for example, mTeSR™1 (Stemcell Technologies, Vancouver, Canada) or Essential 8™ medium (Gibco, Thermo Fisher Scientistic, Waltham, MA) with Geltrex™ (Gibco) coating. In certain embodiments, the cells can be routinely passaged with a cell detachment solution, such as, for example, accutase at ratio of about 1:4 to about 1:6 with 10 μM Y27632, and, optionally, observed daily using microscopy. In certain embodiments, about 1×10⁵ of signalized pluripotent stem cell can be seeded on a cell culture substrate, such as, for example, a Matrigel® (Corning, Corning, NY) coated 6-well plate, optionally coated with about 10 μM Y27632. The feeder-free media can be discarded, and the cells can be washed, preferably at least about 1, 2, 3, 4, 5 or more times in PBS.

In certain embodiments, the pluripotent stem cells can be incubated with a first medium, such as, for example, a neural ectoderm induction medium. In certain embodiments, the first medium comprises a basal medium, such as, for example, Dulbecco's Modified Eagle Medium/Nutrient Mixture F-12 (DMEM: F12) basal medium; a cell culture supplement, such as, for example, an L-alanyl-L-glutamine dipeptide solution (GlutaMAX™, Thermo Fisher Scientific), optionally at a concentration of about 0.01 mM to about 1000 mM, about 0.1 mM to about 100 mM, or about 1 mM to about 10 mM; non-essential amino acids solution, optionally at a concentration of about 0.001 mM to about 100 mM, about 0.01 mM to about 10 mM, or about 0.1 mM to about 1 mM, that can comprise glycine, L-alanine, L-asparagine, L-aspartic acid, L-glutamic acid, L-proline, L-serine, or any combination thereof with each amino acid at a concentration about 1 mM to about 100 mM or about 10 mM in the non-essential amino acids solution; sodium pyruvate, optionally at a concentration of about 0.01 mM to about 1000 mM, about 0.1 mM to about 100 mM, or about 1 mM to about 10 mM; and insulin, transferrin, and selenium preferably provided in a single insulin-transferrin-selenium (ITS) solution in which insulin at a concentration of about 0.1 g/L to about 10 g/L 1 g/L, transferrin is at a concentration of about 0.1 g/L to about 1 g/L or about 0.55 g/L, and sodium selenite is at a concentration of about 0.0001 to about 0.001 g/L or about 0.00067 g/L. The ITS solution can be added at a concentration of about 10 mL for each 1 L of the first medium. The first medium can further comprise about 1 μg/mL to about 100 μg/mL or about 50 μg/mL L-ascorbic acid; an antibiotic solution, such as, for example, a solution comprising penicillin and streptomycin, a GSK-3 inhibitor, such as, for example, about 1 μM to about 100 μM or about 10 μM CHIR-99021; and an inhibitor of the activin receptor-like kinase receptors (e.g., ALK5, ALK4, ALK7), such as, for example, about 1 μM to about 100 μM or about 10 μM SB-431542. In certain embodiments, the culture can be replenished with fresh neural ectoderm induction medium about 1, about 2, about 3, about 4, or more times per day. In certain embodiments, the cells can be cultured for about 1 to about 8 days, preferably about 2 to about 6 days, or, most preferably, about 4 days. In some embodiments, the mesenchymal stem cells can be produced at the conclusion of the culturing with the first medium.

In certain embodiments, first medium can be replaced with a second culture media, such as, for example, mesenchymal stem cells (MSC) culture medium, comprising of a basal medium, such as, for example, alpha-minimum essential medium (MEM) basal medium; about 1% to about 15% or about 10% FBS; a cell culture supplement, such as, for example L-alanyl-L-glutamine dipeptide solution (GlutaMAX™, Thermo Fisher Scientific), optionally at a concentration of about 0.01 mM to about 1000 mM, about 0.1 mM to about 100 mM, or about 1 mM to about 10 mM; about 0.1 ng/mL to about 10 ng/mL or about 1 ng/ml bFGF; about 0.1 ng/mL to about 10 ng/ml or about 1 ng/mL TGFβ1; and an antibiotic solution, such as, for example, a solution comprising penicillin and streptomycin. In certain embodiments, the MSCs can be cultured for about 1 to about 14 days, preferably about 2 to about 12 days, or, most preferably, about 4 to about 10 days. In certain embodiments, obvious fibroblast-like cell morphology can be observed and named passage 0 (P0). In certain embodiments, the cells can be split at a low ratio, such as about 1:2 to about 1:3 at early passages with an enzyme that can dissociate adherent mammalian cells, such as, for example, TrypLE™ (Thermo Fisher) Express on an about 0.1% gelatin coated dish, and after several passage proliferation, MSC can be used for further characterization.

### Methods of Use of the MSCs

In certain embodiments, the MSCs derived from the claim methods can as a model for genetic disorders, such as, for example, Hutchinson-Gilford progeria syndrome (HGPS), including, for example, serving as a cell model for accelerated aging in vitro for mechanistic study and senolytic screening. As reprogramming silenced the LMNA gene in HGPS-iPSCs which is devoid of senescence, it provides an unlimited source for the production of MSCs to serve as a cellular model for HGPS. HGPS is an extremely rare autosomal dominant genetic disorder in which aging associated symptoms are manifested at early stage of life. HGPS provides a useful model for aging study to reveal the underlying molecular mechanism and therapeutic strategies for physiological aging and ageing related diseases. In certain embodiments, to minimize the potential detrimental effects of foreign DNA during the reprogramming, Yamanaka factors can be expressed to avoid insertional mutations in the genome. The HGPS-MSCs derived from the progeria patient manifested irregular nuclear morphology and blabbing, increased DNA damage, defective DNA damage repair, abnormal epigenetic modification, inflammatory cytokines activation and accelerated senescence.

Therefore, in certain embodiments, the subject methods can be used to generate genetically rectified MSCs for autologous stem cell therapy in premature aging. In certain embodiments, LMNA mutation-rectified MSCs from HGPS-iPSCs can be generated. As shown in FIGS. 5A-5J, LMNA mutation-rectified MSCs lost the production of progerin and inflammatory cytokines. Importantly, mutation-corrected MSCs were rejuvenated with a significantly improved survival when transplanted into immunocompromised mice. In certain embodiments, the reprogramming process and MSC induction were carried out with chemically-defined medium without animal components, making it complaint with potential clinical application.

### Materials and Methods

### Primary Cell Isolation and Culture

The dermal tissues were collected from a 5-year-old male HGPS patient and a 26-year-old female normal people through skin biopsy upon the consent given by guardians and human tissue procurement under the guidance of ethical regulations in The Dongguan Women & Children Hospital, CHINA. The dermal tissues were quickly sterilized with 75% ethanol for 1-2 min, washed with 1×PBS buffer twice, excluded extra fat and fascia, and then cut into several 1 mm² pieces before seeding in T25 flask. Five milliliters of fibroblast culture medium containing DMEM/HG, 10% hUCBS, 1 mM GlutaMax, and Penicillin/Streptomycin, was then added to the culture and the expansion of spindle-like fibroblasts was regularly monitored every 24 hrs. Early passages of these cells were cryopreserved with medium plus 10% DMSO. After large-scale cryopreservation, commercial Fetal Bovine Serum (Hyclone) was used to replace hUCBS for the following experiments. All the cell cultures were maintained in a humid incubator at 37° C. with 5% of CO₂.

Human Umbilical Cord Blood Serum (hUCBS) Preparation

Human umbilical cord blood was collected from healthy mothers with no genetic or infectious disease history during childbirth in The Dongguan Women & Children Hospital. After fetal delivery, UCBS was harvested from the umbilical cord vein. The blood was then kept for 2-4 hours at R.T. to precipitate blood cells. After 15 min of centrifugation at 15000 RPM, the serum was carefully isolated, pooled aliquoted, sterilized with a 0.22 μm filter and transferred to −80° C. for long-term preservation.

Minicircle Vector Mediated Human iPSCs Generation

The minicircle DNA mediated reprogramming was performed following previously described⁵¹ with modifications. The minicircle vector MIP247 was a kind gift from Mark Kay & Joseph Wu (Addgene #63726). Early passages of human primary fibroblasts (<passage 10) were cultured as described above and 1×10⁶ of cells were harvested and nucleofected with 15 μg plasmid using commercial kit (Lonza, Cat. No. VPD-1001) under program U-023 before seeding onto two wells of Geltrex™ (Gibco) coated 6-well plate for feeder-free or autogenous feeder cells with fibroblast culture medium. Half of the medium was replenished next day, and 2 mM Valproic Acid (VPA) and 50 μg/mL ascorbic acid were added. On day 3-14, the medium was switched to N2B27 medium containing 50 μg/mL L-ascorbic acid, 50 ng/ml bFGF. Pre-iPSC clones were observed on day 7 and became larger and maturation along with reprogramming process. The medium was replaced with Essential 8 medium for feeder free or knockout serum replacement medium for feeder culture after day 15. The iPSC clones became fully-reprogramed for mechanical picking on day 25-30. At least six different clones of each sample were picked, expanded and cryopreserved. The protocol of iPSCs characterization, such as karyotyping, teratoma formation and embryonic body formation assay is same to previously described 52.

### Confirmation of Minicircle DNA Integration-Free.

Genomic DNA of iPSCs was extracted by phenol chloroform isoamyalcohol extraction and ethanol-precipitation. Minicircle specific primers with fragments homologous to genome DNA were used for PCR and agarose gel electrophoresis. The detailed information of primer sequence is listed in Table 1.

TABLE 1
Summary of the sequence of oligos/primers

| | Oligo sequences from | | Oligo sequences from |
|---|---|---|---|
| Name | 5′ > 3′ | Name | 5′ > 3′ |
| MIP247- | GGCAGAAGGGCAAGAGAA | MIP247- | CTCCCGCCATCTGTTGTTAG |
| Inter-F1 | G (SEQ ID NO: 1) | Inter-R1 | (SEQ ID NO: 2) |
| MIP247- | GTGCCTGGCACCGCCATCA | MIP247- | CGAGAAGCCGCTCCACATAC |
| Inter-F2 | AT (SEQ ID NO: 3) | Inter-R2 | AGT (SEQ ID NO: 4) |
| MIP247- | GACGGCTGTGGCTGGAAGT | MIP247- | CCGCTTGGCCTCGTCGATGA |
| Inter-F3 | TCG (SEQ ID NO: 5) | Inter-R3 | A (SEQ ID NO: 6) |
| MIP247- | GACAGGTGCCTCTGCGGCC | MIP247- | CGTACGCCTTGGAGCCGTAC |
| Inter-F4 | AA (SEQ ID NO: 7) | Inter-R4 | A (SEQ ID NO: 8) |
| HGPS-GT- | CTAGCGAGGGCCTATTTCC | pG13.0- | TGTCTCGAGGTCGAGAATTC |
| F1 | CATG (SEQ ID NO: 9) | sgRNA-R | (SEQ ID NO: 10) |
| Sox2-qRT- | AGCTACAGCATGATGCAG | Sox2-qRT- | GGTCATGGAGTTGTACTGCA |
| F | GA (SEQ ID NO: 11) | R | (SEQ ID NO: 12) |
| Nanog-qRT- | TGAACCTCAGCTACAAACA | Nanog-qRT- | TGGTGGTAGGAAGAGTAAA |
| F | G (SEQ ID NO: 13) | R | G (SEQ ID NO: 14) |
| Rex1-qRT- | TCGCTGAGCTGAAACAAAT | Rex1-qRT- | CCCTTCTTGAAGGTTTACAC |
| F | G (SEQ ID NO: 15) | R | (SEQ ID NO: 16) |
| Klf4-qRT- | TCTCAAGGCACACCTGCGA | Klf4-qRT- | TAGTGCCTGGTCAGTTCATC |
| F | A (SEQ ID NO: 17) | R | (SEQ ID NO: 18) |
| Myc-qRT- | ACTCTGAGGAGGAACAAG | Myc-qRT- | TGGAGACGTGGCACCTCTT |
| F | AA (SEQ ID NO: 19) | R | (SEQ ID NO: 20) |
| DNMT3B- | AAGCTACACACAGGACTTG | DNMT3B- | AGTTCGGACAGCTGGGCTTT |
| qRT-F | ACAG (SEQ ID NO: 21) | qRT-R | (SEQ ID NO: 22) |
| TERT-qRT- | TGTGCACCAACATCTACAA | TERT-qRT- | GCGTTCTTGGCTTTCAGGAT |
| F | G (SEQ ID NO: 23) | R | (SEQ ID NO: 24) |
| GDF3-qRT- | AAATGTTTGTGTTGCGGTC | GDF3-qRT- | TCTGGCACAGGTGTCTTCAG |
| F | A (SEQ ID NO: 25) | R1 | (SEQ ID NO: 26) |
| 18S-rRNA- | GTAACCCGTTGAACCCCAT | 18S-rRNA- | CCATCCAATCGGTAGTAGCG |
| qRT-F | T (SEQ ID NO: 27) | qRT-R | (SEQ ID NO: 28) |
| GAPDH- | CAAAGTTGTCATGGATGAC | GAPDH- | CCATGGAGAAGGCTGGGG |
| qRT-F | C (SEQ ID NO: 29) | qRT-R | (SEQ ID NO: 30) |
| LaminB1- | GCTGCTCCTCAACTATGCT | LaminB1- | GAATTCAGTGCTGCTTCATA |
| qRT-F | AAG (SEQ ID NO: 31) | qRT-R | TTCTC (SEQ ID NO: 32) |
| LaminB2- | AGTTGGACGAGGTCAACA | LaminB2- | GGACTCCAGGTCCTTCACAC |
| qRT-F | AGAG (SEQ ID NO: 33) | qRT-R | (SEQ ID NO: 34) |
| LC-qRT- | CTCAGTGACTGTGGTTGAG | LC-qRT- | AGTGCAGGCTCGGCCTC |
| F | GA (SEQ ID NO: 35) | R | (SEQ ID NO: 36) |
| Progerin- | CTCAGTGACTGTGGTTGAG | Progerin- | TTCTGGGGGCTCTGGGCT |
| qRT-F | GA (SEQ ID NO: 37) | qRT-R | (SEQ ID NO: 38) |
| hLA-Exon1- | AATGATCGCTTGGCGGTCT | LA-Exon1- | CACCTCTTCAGACTCGGTGA |
| qRT-F1 | AC (SEQ ID NO: 39) | qRT-R1 | T (SEQ ID NO: 40) |
| LaminA- | CTGCTTCCAGGAAACTCCA | LaminA- | GCCAGGGCAGAAAAGCAGA |
| qRT-F1 | C (SEQ ID NO: 41) | qRT-R1 | AG (SEQ ID NO: 42) |
| OCT4-BS- | TTGGGATGTGTAGAGTTTG | OCT4-BS- | TAAACCAAAACAATCCTTCT |
| F1 | AGA (SEQ ID NO: 43) | R1 | ACTCC (SEQ ID NO: 44) |
| AP2a-qRT- | ATTGACCTACAGTGCCCAG | AP2a-qRT- | ATGCTTTGGAAATTGACGGA |
| F1 | C (SEQ ID NO: 45) | R1 | (SEQ ID NO: 46) |
| HNK1-qRT- | CGGAAGCAGGTTTGGAGA | HNK1-qRT- | CGGAGACGCTCCGGACT |
| F1 | (SEQ ID NO: 47) | R1 | (SEQ ID NO: 48) |
| p75-qRT- | CAGGCTTTGCAGCACTCAC | p75-qRT- | CTGCTGCTGTTGCTGCTTCT |
| F1 | (SEQ ID NO: 49) | R1 | (SEQ ID NO: 50) |
| PAX3-qRT- | GCATGTTCAGCTGGGAAAT | PAX3-qRT- | ATGCTGTGTTTGGCCTTCTT |
| F1 | C (SEQ ID NO: 51) | R1 | (SEQ ID NO: 52) |
| SOX10- | CTTTCTTGTGCTGCATACG | SOX10- | AGCTCAGCAAGACGCTGG |
| qRT-F1 | G (SEQ ID NO: 53) | qRT-R1 | (SEQ ID NO: 54) |
| ZIC1-qRT- | CACGTGCATGTGCTTCTTG | ZIC1-qRT- | GCGCTCCGAGAATTTAAAGA |
| F1 | (SEQ ID NO: 55) | R1 | (SEQ ID NO: 56) |
| SOX9-qRT- | AGCGAACGCACATCAAGA | SOX9-qRT- | CTGTAGGCGATCTGTTGGGG |
| F1 | C (SEQ ID NO: 57) | R1 | (SEQ ID NO: 58) |
| p16-qRT- | CGGTCGGAGGCCGATCCA | p16-qRT- | GCGCCGTGGAGCAGCAGCA |
| F1 | G (SEQ ID NO: 59) | R1 | GCT (SEQ ID NO: 60) |
| p21-qRT- | CCTGTCACTGTCTTGTACC | p21-qRT- | GCGTTTGGAGTGGTAGAAAT |
| F1 | CT (SEQ ID NO: 61) | R1 | CT (SEQ ID NO: 62) |
| IL1α-qRT- | GTGCTGCTGAAGGAGATGC | IL1α-qRT- | CCCCTGCCAAGCACACCCAG |
| F1 | CTGA (SEQ ID NO: 63) | R1 | TA (SEQ ID NO: 64) |
| IL1β-qRT- | TGCACGCTCCGGGACTCAC | IL1β-qRT- | CATGGAGAACACCACTTGTT |
| F1 | A (SEQ ID NO: 65) | R1 | GCTCC (SEQ ID NO: 66) |
| IL6-qRT- | CCAGGAGCCCAGCTATGA | IL6-qRT- | CCCAGGGAGAAGGCAACTG |
| F1 | AC (SEQ ID NO: 67) | R1 | (SEQ ID NO: 68) |
| IL8-qRT- | GAGTGGACCACACTGCGCC | IL8-qRT- | TCCACAACCCTCTGCACCCA |
| F1 | A (SEQ ID NO: 69) | R1 | GT (SEQ ID NO: 70) |
| PAI-1-qRT- | CCTGGCCTCAGACTTCGGG | PAI-1-qRT- | GGGGCCATGCCCTTGTCATC |
| F1 | GT (SEQ ID NO: 71) | R1 | AAT (SEQ ID NO: 72) |

### Bisulfite Genomic Sequencing

Bisulfite treatment was carried out using the DNA Bisulfite Conversion Kit (TIANGEN BIOTECH). Converted DNA was used as template for PCR to analyze DNA methylation state of endogenous OCT4 promoter region using primers in oligo tables. PCR was performed using PrimeSTAR (Takara) and PCR products were purified and cloned into pJET1.2 vector. Sequences of 10 random picked bacterial clones were analyzed. Each column of circles for a given amplicon represents the methylation status of CpG dinucleotides in one clone for that region. Open circles are unmethylated CpGs and closed circles methylated ones. The left numbers of each column indicate CpG localization relative to the transcriptional start site. The detailed information of primer sequence is listed in Table 1.

### Reverse-Transcription PCR and Quantitative PCR

RNA was extracted from culture cells by Trizol (Sigma) and 1 μg RNA was used for cDNA synthesis. For reverse PCR, cDNAs were diluted to 200-fold as template and high-fidelity DNA polymerase was used. For qPCR, SYBR Green PCR Kit (Applied Biosystems) was used with cDNAs diluted 50-fold as template and PCR was carried out at standard thermal cycling conditions (95° C. for 20 s; 40 cycles 95° C. for 20 s; 60° C. for 30 s. For melting curve, 95° C. for 15 s; 60° C. for 1 minute; 95° C. for 15 s), three different repeats were performed and ΔΔCT values were calculated for statistical analysis. The detailed information of primer sequence is listed in Table 1.

### Immunostaining

The cells were blocked with blocking buffer (PBST containing 5% FBS and 5% BSA) for 1 hour after fix and permeabilization before incubated with primary antibodies at 4° C. overnight. After washing with PBST, the cells were incubated in secondary fluorescent antibodies for 1 hour and counterstained with DAPI (Life Technology). Images were acquired with the confocal microscope (Carl Zeiss LSM800) and were processed with ZEN Blue software. For DNA damage response assay, cells were exposed to 10 Gy γ-irradiation. The detailed information of antibodies used are given in Table 2.

TABLE 2
Summary of antibodies used.

| | | | | WB | IF |
|---|---|---|---|---|---|
| Antibody | Species | Vendor | Cat. NO. | dilution | dilution |
| HP1 alpha | Rb | Abcam | ab109028 | 1/3000 | 1/250 |
| H3K9me3 | Rb | Abcam | ab176916 | N/A | 1/200 |
| H3K27me3 | Rb | Abcam | ab6002 | N/A | 1/200 |
| SOX10 | Rb | Abcam | ab155279 | 1/1500 | 1/100 |
| Nestin | Rb | Beyotime | AF2215 | N/A | 1/50 |
| LMNB2 | Rb | Beyotime | AF0219 | 1/1000- | 1/200 |
| | | | | 1/5000 | |
| Lamin B1 | Rb | Beyotime | AF1408 | 1/1500 | N/A |
| Lamin B1 | Rb | Proteintech | 12987-1-AP | 1/2000 | 1/50 |
| Lamin A/C | Rb | Proteintech | 10298-1-AP | 1/1000 | 1/100 |
| Nanog | Ms | Santa Cruz | sc-374001 | N/A | 1/50 |
| Oct-3/4 | Ms | Santa Cruz | sc-5279 | N/A | 1/50 |
| SSEA-4 | Ms | Santa Cruz | sc-21704 | N/A | 1/50 |
| TRA-1-60 | Ms | Santa Cruz | sc-21705 | N/A | 1/50 |
| Progerin | Ms | Sigma | SAB4200272 | 1/3000 | 1/200 |
| γH2A.X | Ms | Millipore | 05-636 | N/A | 1/100 |
| 53BP1 | Rb | Santa Cruz | sc-22760 | N/A | 1/50 |

### Cell Lysis and Western Blot

The cells were lysed in RIPA buffer containing protease inhibitor (Roche) and incubated on ice for 30 minutes. Cell lysate was then centrifuged 1,3000 rpm at 4° C. and the soluble proteins were boiled with loading dye before subjected to electrophoresis in 8% SDS-PAGE gel for separation and western blotting.

MSCs generation via temporal neutralized ectoderm induction of hPSCs hPSCs is routinely cultured in mTeSR1 or Essential 8 media with splitting at 1:4-1:6 every 3 days. For neutralized ectoderm differentiation, 1×10\hat{ }5 of signalized hPSCs were seeded on Matrigel coated 6-well plate with 10 μM Y27632. The medium was discarded, washed with DMEM: F12 twice and changed to neural ectoderm induction medium, consisting of 50% Neurobasal, 50% DMEM/F12 medium, 1% N2 and 2% B27 and 3-6 μM CHIR-99021 and 10 μM SB-431542. Replenish the medium daily for 5 days until the cells exhibited neural-like morphology, spread out and separated from each other. Signalized the cells and re-seeding them on Matrigel-coated plate with MSC culture medium, consisting of alpha-MEM basal medium, 10% FBS, 1× Glutamax solution, 5 ng/ml bFGF, 5 ng/ml EGF and 1× Penicillin-Streptomycin. After additional 5-7 days of induction, fibroblast-like morphology is observed. Split the cell at low ratio, such as 1:2-1:3 at early passages with TrypLE Express. Flow cytometry analysis to characterize MSC surface markers are usually performed at passage 3-5.

### Flow Cytometry Analysis of MSCs Surface Markers

When MSCs reached 80-90% confluency, cells were washed with 1×PBS, dissected to single cells, and finally resuspended in 1.2 ml of 1×PBS containing 3% FBS. Cells were aliquoted equally to six different tubes and stained with fluorescent antibodies (BD 562245) for 30 min protected from light. Cells were washed twice with 1×PBS and resuspended with 150 μl 1×PBS containing 3% FBS and transferred to Flow Cytometry Tubes for BD FACSAria SORP analysis. Data were analyzed with FlowJo 10.7.

### Tri-Lineage Differentiation of MSCs

Adipogenesis. For adipocyte differentiation, cells growing to 90-100% confluency were washed with 1×PBS for three times before switched to adipocyte induction medium, consisting of HG-DMEM basal medium supplemented with 10% FBS, 0.5 mM 3-isobutyl-1-methylxanthine (IBMX), 100 μM Indomethacin, 1 μM Dexamethasone, 10 μg/ml Insulin and 1× Penicillin-Streptomycin solution, with medium change every 3 days. After 21 days of induction, cells were fixed in 4% paraformaldehyde and stained with Oil Red O solution.

Osteogenesis. For osteoblast differentiation, cells growing to 90-100% confluency were washed with 1×PBS for three times before switched to osteoblast induction medium, consisting of HG-DMEM basal medium supplemented with 10% FBS, 50 μg/ml L-Ascorbic acid, 0.1 μM Dexamethasone, 10 mM β-glycerophosphate, 1 mM Sodium pyruvate and 1× Penicillin-Streptomycin solution, with medium change every 3 days. After 21 days of induction, cells were fixed in 4% paraformaldehyde and stained with Alizarin Red S solution.

Chondrogenesis. For chondrocyte differentiation, cells growing to 90-100% confluency were washed with 1×PBS for three times before switched to chondrocyte induction medium, consisting of LG-DMEM basal medium supplemented with 10% FBS, 50 μg/ml L-Ascorbic acid, 0.1 μM Dexamethasone, 10 ng/ml TGFβ3, 1 mM Sodium pyruvate, 1× Insulin-Transferrin-Selenium solution and 1× Penicillin-Streptomycin solution, with medium change every 3 days. After 21 days of induction, cells were fixed in 4% paraformaldehyde and stained with Alcian blue solution.

### CRISPR/Cas9-Based Double Selection Strategy or Mutation Correction

The gene-editing associated plasmids were ordered from Addgene, eSpCas9 (1.1) (#71814) and pGL3.0-sgRNA (#51133). To exclude the possibility of gene editing caused side-effects, small guide RNA (sgRNA) was designed to target noncoding regions, outside of human LMNA genome locus. sgRNA was designed with Benchling (see worldwide website: benchling.com) according to Sp-Cas9 PAM (NGG) module and on/off target scores. Oligos were synthesized from IDT and annealed into pGL3.0-sgRNA vector. For the donor vector, left and right homologous arms were amplified from wild-type H9 genome DNA with 3.5 kb and 1.2 kb, respectively, and assembled with lox P-PGK-Puro-T2A-mCherry-lox P fragment via Gibson assembly. Plasmid sequence was confirmed with sanger sequencing. Total 30 μg of plasmids Cas9: sgRNA: donor vector (4:1:5) were delivered to iPSCs with nucleofection using program B-16 (Amaxa nucleofector). The cells were selected using 500 ng/ml puromycin 48 hours post nucleofection for a week and seeded at low-density to Matrigel-coated dish for single cell colonies picking. Puro-mCherry double labelling clones were picked, transferred to 48-well plate, and expanded for genome typing. HGPS mutant region was amplified by PCR using Taq DNA Polymerase (TAKARA) and mutation correction were checked with sanger sequencing. The corrected clones only exhibited a unique C in LMNA c.1824C.>T but not C/T signal. The corrected single clone (HGPS-CiPSC) was further transduced with pCAG-Cre-2A-GFP to remove PGK-Puro-T2A-mCherry double labelling cassette. The mCherry negative cells were collected as desired HGPS-CiPSC with FACS sorting.

### Senescence β-Galactosidase Staining

SA-b-gal staining was performed using a commercial kit (Beyotime Biotechnology) according to instruction manual. In brief, cells were fixed in fixative at RT for 5 min. After fixation, cells were stained with freshly prepared staining solution at 37° C. overnight. Images were taken and the percentage of senescent cells were analysed by ImageJ for quantitative analysis.

### In Vivo Bioluminescence of MSCs

Lentivirus vector containing human PGK promoter driven luciferase gene cassette was packaged by co-transfection psPAX2 and pMD2.G into HEK293T and the virus was concentrated by ultracentrifuge 35,000 RPM for 3 hrs. MSCs was infected with the virus and were selected with 10 ng/ml Blasticidin S after 48 hours of incubation. The stable MSC-luciferase cells were expanded and harvested for in vivo injection. About 1×10\hat{ }6 cells were re-suspended in 100 μl PBS and injected into midportion of the tibialis anterior (TA) muscle of nude mouse. Mouse were anaesthetized and also treated with D-luciferin (150 μg/g ratio) every other day. Photon emission was detected and measured using IVIS lumina system. Bioluminescence images were acquired and quantified for statistical analysis.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Generation and Characterization of Human iPSC Via Integration-Free Minicircle Vector

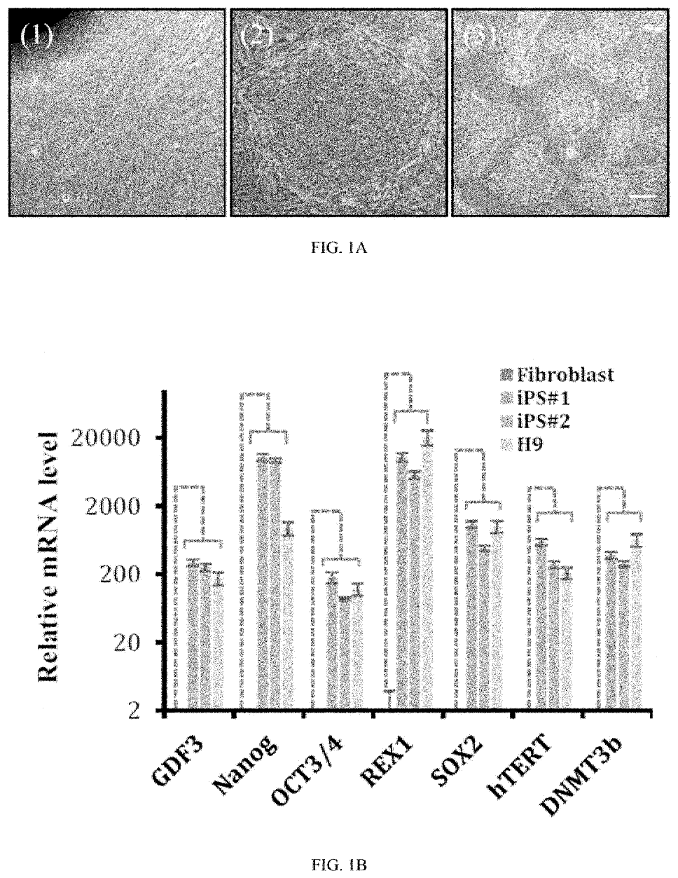

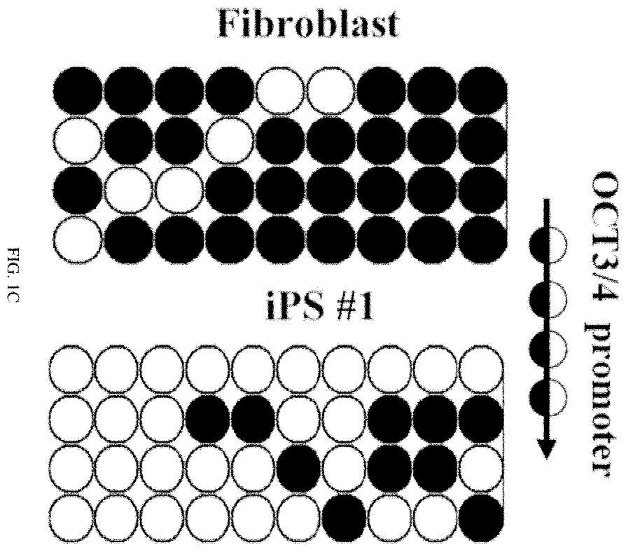

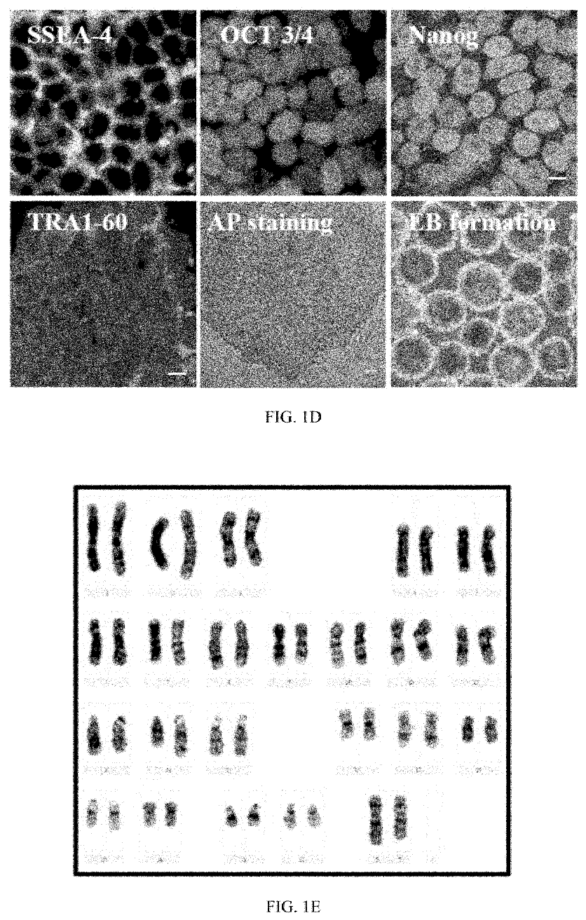

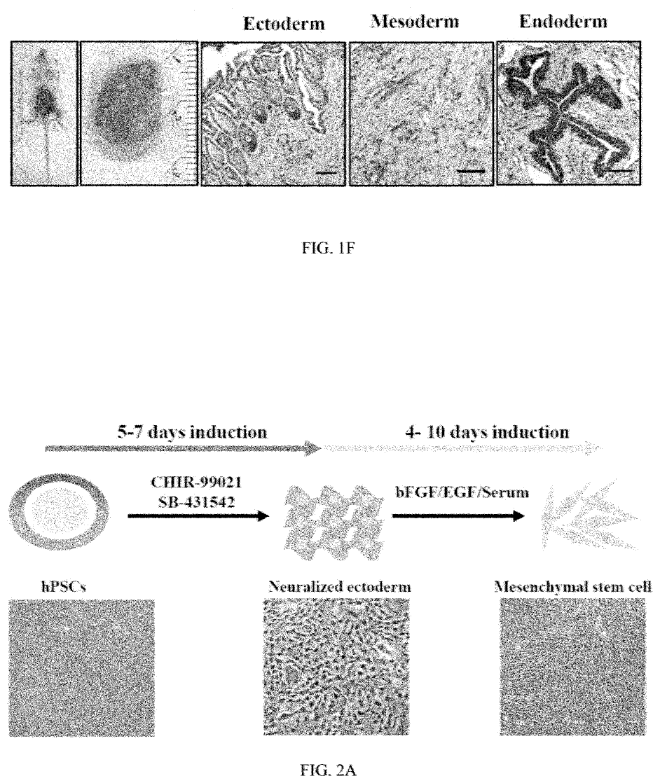

Although somatic cell nuclear transfer (SCNT) mediated generation of ESCs and animal cloning have achieved remarkable progress in animals and human, ethical issues remain as an obstacle in clinical applications. Patient-specific iPSCs have similar potential as ESCs for disease modeling, drug screening and regenerative medicine without much ethical concerns, providing a better source for personalized stem cell applications. However, majority of the strategies in iPSC derivation are based on viral integration which may lead to oncogenic mutations. To address the safety concerns, we employed a minicircle vector reported previously to generate human iPSC from primary cells (FIG. 6A). Dermal skin from a healthy donor was used for fibroblast preparation. To minimize the impact of cellular senescence on reprogramming,²⁷ we started the nucleofection with MIP 247 vector in fibroblasts within passage 10. The process of reprogramming is illustrated in (FIG. 6A). At least six independent iPSCs clones were collected and expanded on either autologous feeder-layer or Matrigel (FIG. 1A). Alternatively, we also used renal epithelial cells collected from urine to derive iPSCs to avoid the invasive approach in biopsy (FIG. 6C).

To characterize these iPSCs, genomic DNA were extracted to confirm the absence of minicircle plasmid insertion with four pairs of primers (FIG. 6B). As shown in (FIG. 6D), neither iPSC clones nor the H9 hESCs were positive for vector insertion. The iPSCs expressed pluripotency markers, such as GDF3, OCT3/4, NANOG, REX1, SOX2, and DNMT3b, at levels comparable to that in human ESC H9 (FIG. 1B). Bisulfite sequencing at OCT3/4 locus in the genomic DNA revealed a significant reduction in DNA methylation in iPSCs (FIG. 1C), indicating epigenetic memory has been erased during reprogramming. The pluripotency was also confirmed by immunostaining of SSEA4, OCT3/4, NANOG, TRA1-60, and Alkaline Phosphatase (AP) activities, as well as the capability of in vitro embryoid body (EB) formation (FIG. 1D). Genome integrity was shown by normal chromosome pairing (44+XX) in karyotyping examination (FIG. 1E). In addition, when injected into immunocompromised mice, these iPSCs gave rise to three germ layers demonstrated by the hematoxylin and eosin (H&E) staining in the teratoma formation assay (FIG. 1F). Taken together, iPSCs generated from human primary cells are footprint free and clinically compliant, given that no animal components were used in the primary cell culture and reprogramming process.

### Example 2—Generation of Mesenchymal Stem Cells from Human Pscs by a Two-Stage Induction Approach

MSCs can be induced from hPSCs through an intermediate stage, the NCCs. 24-26. 28-30 However, the efficiency of NCCs induction from hPSCs is relatively low. The use of flow cytometric selection for HNK1/p75⁺⁺ to purify NCCs is not practical for large-scale production of MSCs with a possibility of introducing undesired contamination.^{25, 31} Recently, Menendez et al. reported that over 85% pure NCCs can be generated directly from hPSCs without FACS sorting. However, the in-house made culture media for hPSCs and for NCCs induction are complicated. The entire process of NCCs induction takes more than 20 days 26 and two extra weeks are then required for transition from NCCs to MSCs. Given that 1) NCCs from neural ectoderm are easily transdifferentiated into MSCs with high efficiency; 2) non-NCC cells derived from neural ectoderm, such as neurons and astrocytes, are more hypersensitive to stress than MSCs, it is plausible that MSCs can be directly and rapidly induced from neural ectoderm cells without NCC purification.

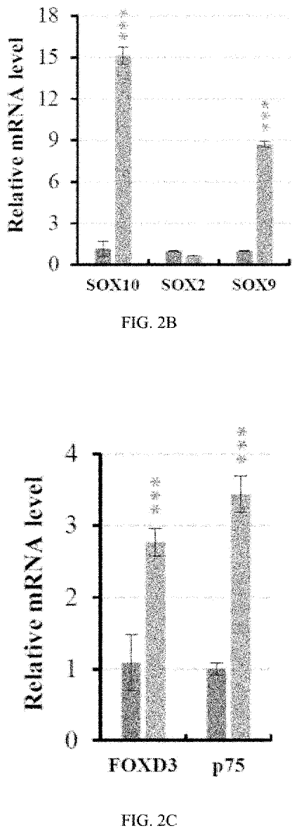

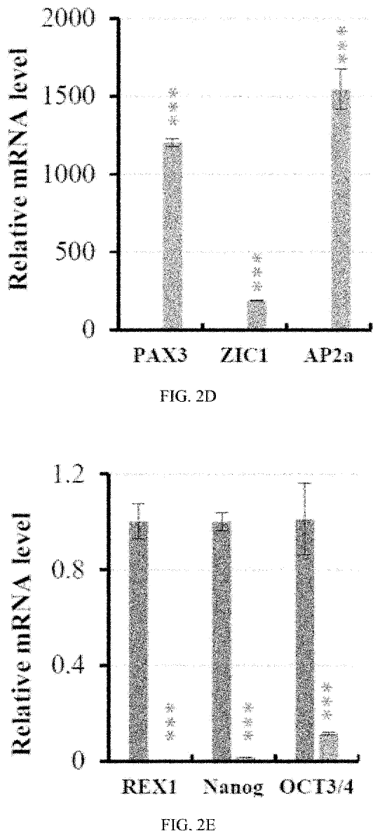

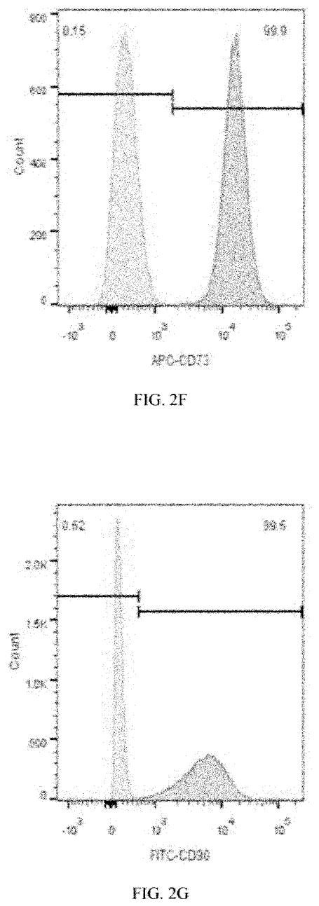

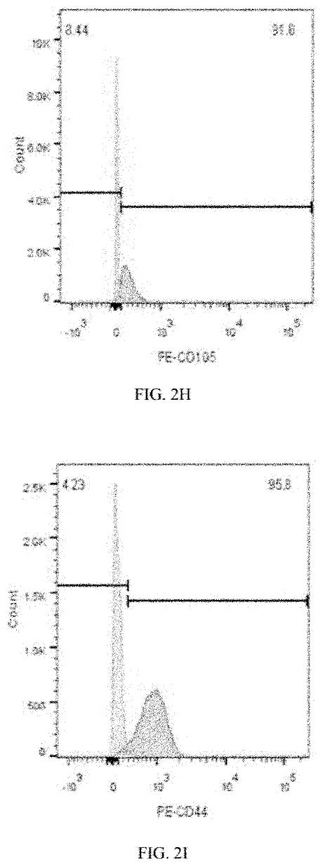

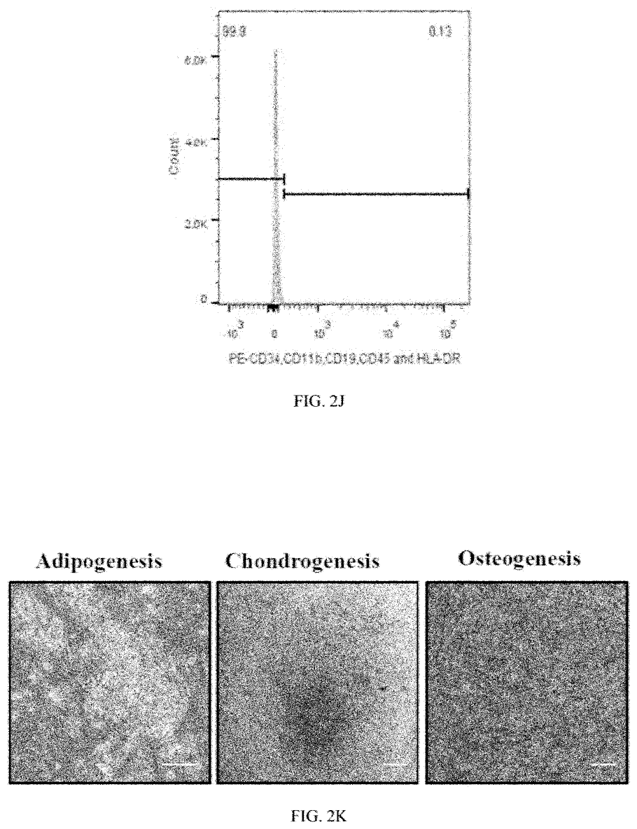

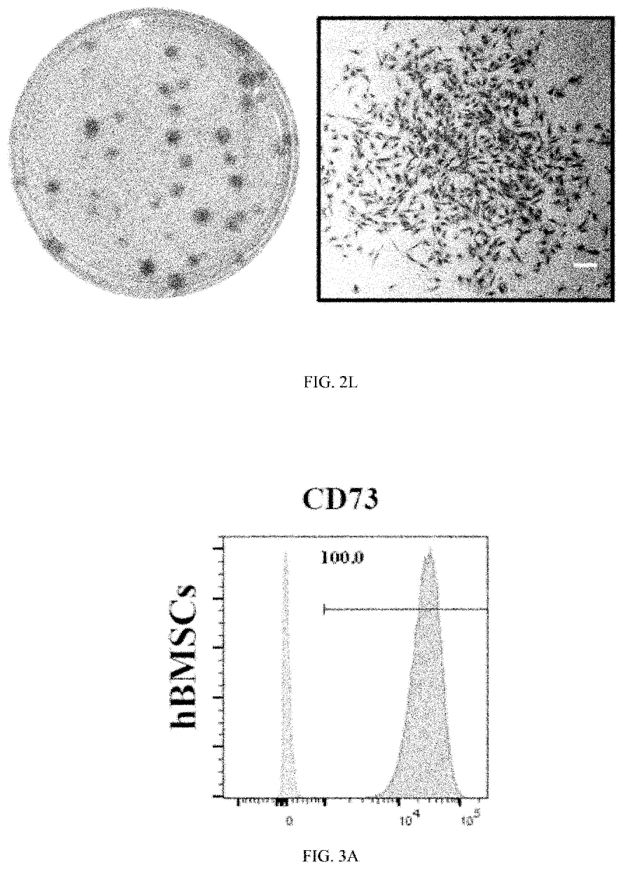

To verify the hypothesis, we firstly induced hPSCs to neural ectoderm using modified medium composed of 48.5% of Neurobasal medium, 48.5% of DMEM/F12 basal medium, 1% of N₂ and 2% of B27, 10 μM of SB431542 (TGF-β pathway inhibitor) and 3 μM of ChIR99021 (WNT signal activator). After induction for 5 days, cells exhibited neural-like morphology, spreading out and separating from each other, though small patches of hPSCs-like clusters may still be observed (FIG. 2A). The qPCR analysis revealed that OCT3/4, NANOG, and REX1 were dramatically decreased while neural ectoderm-associated genes including PAX3, ZIC1, SOX10, SOX9, AP2a, FOXD3 and p75, were upregulated (FIGS. 2B-2E). In line with this, immunostaining showed loss of pluripotent markers OCT3/4, NANOG and AP2α and expression of PAX7 and typical neural progenitor/stem cell marker NESTIN (FIG. 7C). The induced neural ectoderm cells were capable of suspension culture (FIG. 7A). p75 has been identified as one of the MSCs markers in vivo. 32 Ninety-nine percent of neural ectoderm cells were found positive for p75 (FIG. 7B). However, the percentage of p75^{high} was lower compared with previous study.²⁶ Upon culture for two weeks, these neural ectoderm cells were able to be differentiated into adipocytes, chondrocytes, and osteocytes in corresponding induction media (FIG. 7D). Collectively, these data suggest that these neural ectoderm cells are not pure NCCs, rather populations manifesting MSCs-like properties.

To induce MSCs from neural ectoderm cells, we switched the culture medium directly to MSC medium containing 10% of serum supplemented with 2 ng/ml of bFGF and 2 ng/ml of EGF. To generate clinical compliant MSCs, human umbilical cord blood serum (hUCBS) was used in the medium instead of FBS during the induction of MSCs. Spindle-like morphology was initially observed on day 4 after medium switch. Cells were split at a ratio 1:1 or 1:2 when reached full confluency. Moderate cell death was observed at the first two passages. After three passages, the cells became homogenous with enhanced proliferation rate. Flow cytometry analyses showed over 90% of the cells were positive for typical MSC surface markers (CD73 99.9%, CD90 99.5%, CD105 91.6% and CD44 95.8%) and negative for CD45, CD34, CD11b, CD19 and HDL-DR (<1%) (FIGS. 2F-2J). To examine if these MSCs retain full differentiation potentials, MSCs at passage 5 were induced for adipogenesis, chondrogenesis and osteogenesis. As shown in FIG. 2L, differentiated cells were stained positively by Oil red O, Alcian blue, and Alizarin red (FIG. 2K). Colony formation assay showed a strong self-renewal capacity of the neural ectoderm cells-derived MSCs (FIG. 2L).

### Example 3—HPSCs-Derived MSCs Exhibit Similar Properties and Gene Expression Profile as Bone Marrow-Derived MSCs

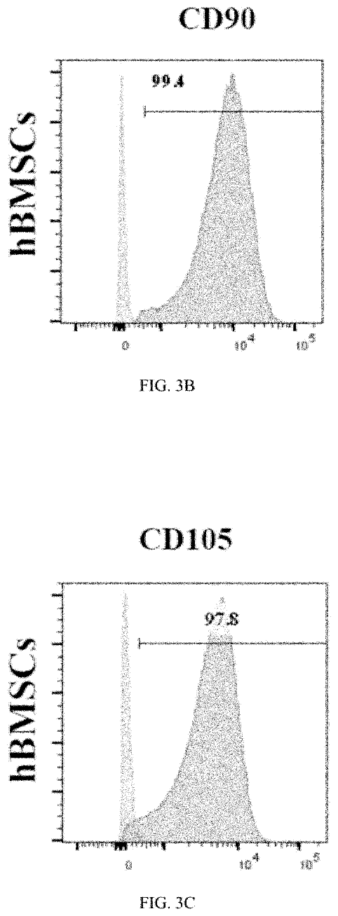

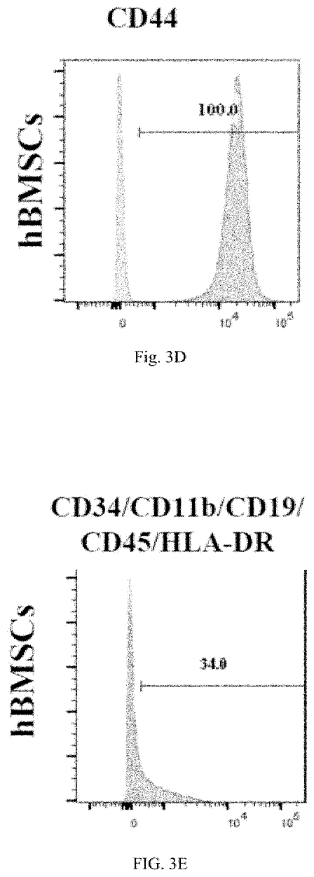

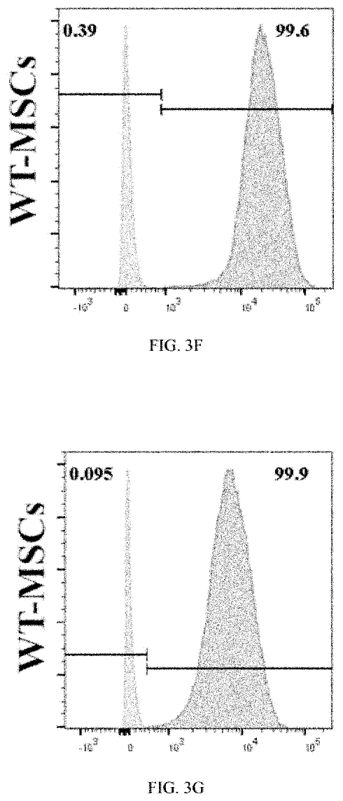

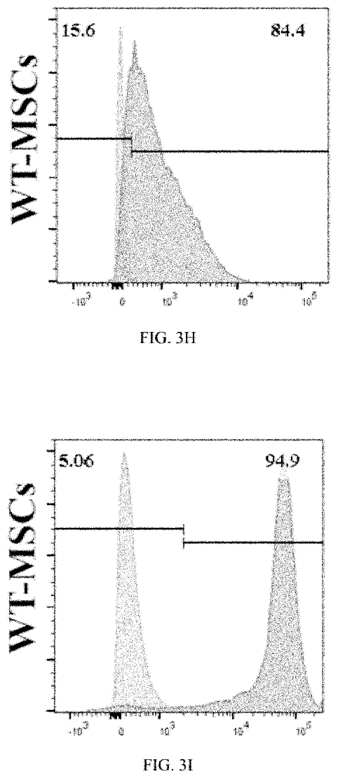

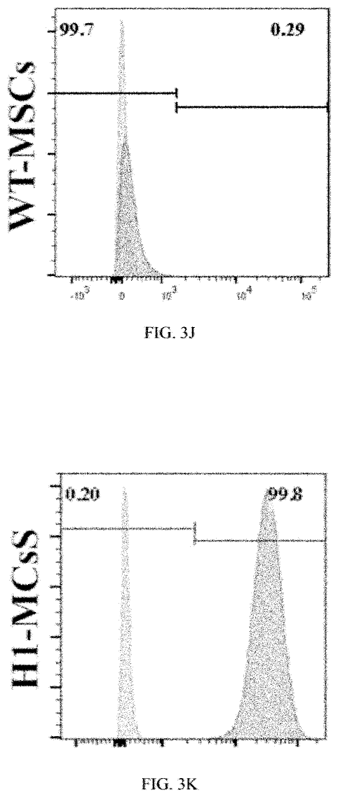

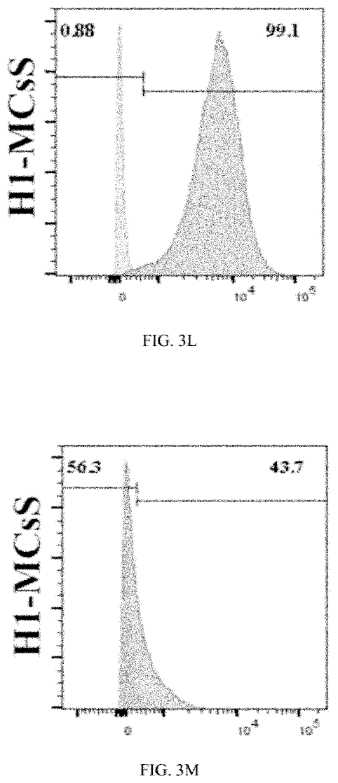

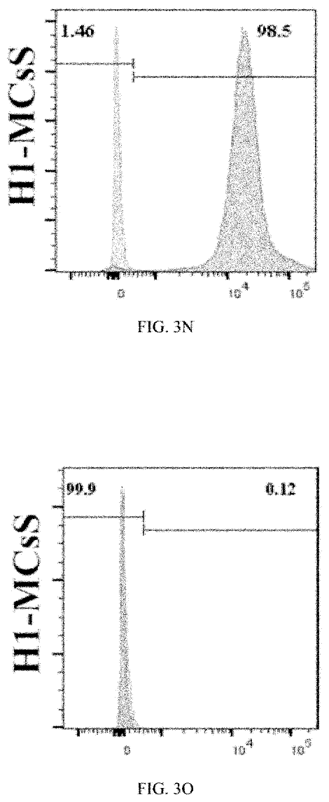

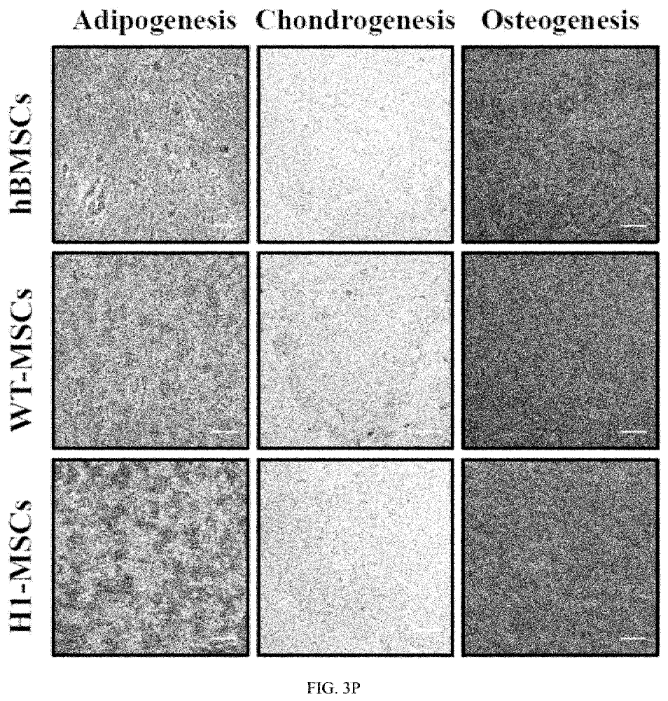

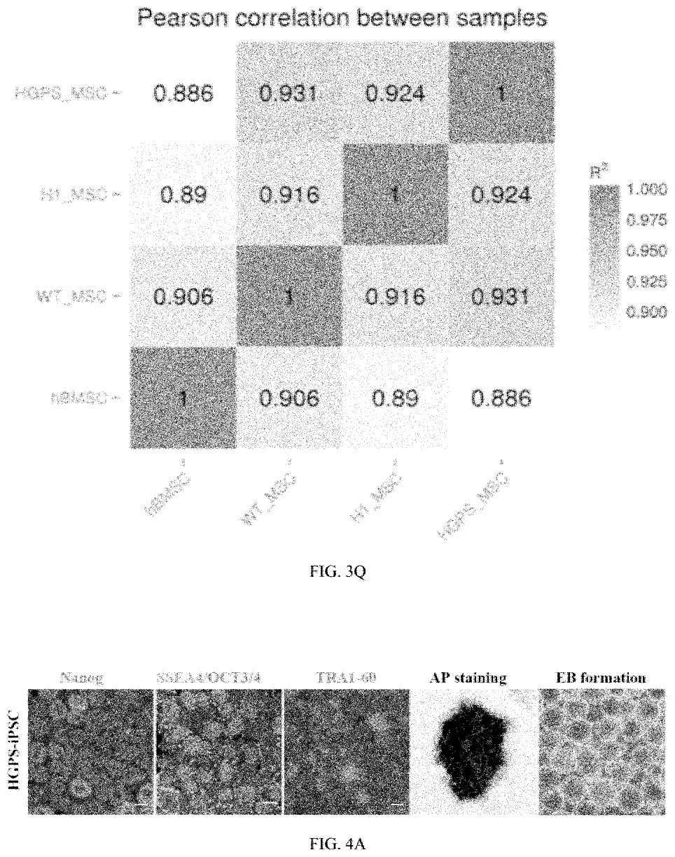

Differences have been reported in the efficiency of MSC induction between human ESCs and iPSC. It is in general easier for ESCs than iPSCs to differentiate into MSCs as the later require longer time and specific stress adaptation.^{21, 22} To investigate whether the reduced efficiency of differentiation in iPSCs is a result of the clonal variation of iPSC quality, we derived MSCs from the second human iPSCs clone (WT-MSCs) and human ESCs H1 (H1-MSCs). Interestingly, the iPSCs and ESCs showed comparable differentiation efficiencies in our hands and similar MSCs surface markers including CD73, CD90, and CD44, with an exception for CD105 exhibiting higher percentage in iPSC-derived WT-MSCs (84%) than in H1-MSCs (43%) (FIGS. 3A-30). The variable percentage of CD105 is in line with the previously reported NCCs-induced MSCs.^{33,34} Overall, these results demonstrated that MSCs could be robustly and efficiently generated within a substantially shorter time from both human ESCs and iPSCs by our protocol.

To explore the similarity and difference between hPSCs-derived MSCs and mature MSCs directly isolated from tissues, we took primary bone marrow MSCs (BM-MSCs) for comparison. BM-MSCs exhibited high percentage of surface markers CD73, CD90, CD105 and CD44. However, a sub-population of these BM-MSCs were also positive for CD34, CD11b, CD19, CD45 and HLA-DR cocktails (FIGS. 3A-30). When differentiation capabilities were examined, adipocyte, chondrocytes and osteoblasts were all successfully induced by BM-MSC, ESCs-MSCs and iPSCs-MSCs verified by staining of Oil Red O, Alcian Blue and Alizarin Red S (FIG. 3P). Of note, the oil droplets in BM-MSCs was found significantly larger than that observed in H1-MSCs or WT-MSCs (FIG. 3P), which is consistent with the previous reports.²²⁻²⁴ To further compare these MSCs generated from different sources, gene expression profiles were analyzed. As shown in FIG. 3Q, a high correlation co-efficient (R2) was observed between BM-MSCs and either WT-MSCs (90.6%) or H1-MSCs (89.0%), suggesting that both hPSCs-derived MSCs and ESCs-derived MSCs are highly similar to primary BM-MSCs. The hPSCs-derived MSCs appeared to be more homogenous with 91.6% similarity (H1-MSCs vs WT-MSCs) (FIG. 3Q). While BM-MSCs went cell cycle arrest after passage 5, hESCs derived MSCs did not show cellular senescence before passage 20.

### Example 4—Generation and Characterization of iPSCS from a HGPS PATIENT

The human progeroid syndrome Hutchinson-Gilford progeria syndrome (HGPS) is predominantly caused by an autosomal dominant mutation (p.G608G, c.1824C.>T) in exon 11 of LMNA gene, resulting in the exposure of a cryptic splicing site and generation of a truncated mutant lamin A isoform, termed progerin. Progerin is an immature truncated lamin A missing 50 amino acids covering the second cleavage site. It is permanently farnesylated, therefore tightly attaches to the nuclear envelope, leading to the deformation of nuclear architecture, genome instability and epigenetic alternations.³⁵ Progerin expression is also observed in the elderlies 36 and ectopic expression of progerin in normal cells recapitulates accelerated cellular senescence in vitro and results in the collapse of tissue homeostasis in vivo.³⁷⁻³⁹ Interestingly, neural cells of HGPS patients are unaffected, whereas mesoderm linages, especially mesenchymal cells, are severely affected,⁴⁰⁻⁴² implicating HGPS-MSCs as an ideal cell model for stem cell aging and drug screening.

We firstly generated iPSCs using dermal fibroblasts derived from a HGPS patient. The expression of progerin were confirmed in HGPS fibroblasts by Western blotting using antibodies against lamin A/C (FIG. 4D). To generate iPSC cell lines, HGSP fibroblasts at passage 5 were reprogrammed with a minicircle vector expressing 4 Yamanaka factors. Valproic acid and ascorbic acid were supplemented in the culture medium to improve the reprogramming efficiency. Among six HGPS-iPSC clones, two were chosen for further characterization. No detectable integration of vector fragments into iPSC genome was observed in both clones (FIG. 8A).

Similar to H9 ESCs, both HGPS-iPSCs clones expressed pluripotency associated markers, such as GDF3, NANOG, OCT3/4, SOX2, hTERT and DNMT3b at mRNA level (FIGS. 8B-8C). Immunostaining and immunochemistry confirmed the expression of NANOG, SSEA4, OCT3/4, TRA1-60, and AP activity in HGPS-iPSCs. In addition, reprogrammed HGPS-iPSCs can form EB (FIG. 4A). Consistent with previous reports, the transcription of lamin A, lamin C or progerin were silenced whereas lamin B1 mRNA was upregulated significantly in HGPS-iPSCs, compared with the parental fibroblasts (FIG. 8D). Bisulfite sequencing revealed that the endogenous OCT3/4 promoter region was in an open status in HGPS-iPSCs (FIG. 4B). Karyotyping and in vivo teratoma formation assay showed that HGPS-iPSCs retained the genomic integrity (44+XY) with full differentiation potential (FIGS. 3E-3F). HGPS cells, including fibroblasts, and smooth muscle cells (SMCs) and MSCs manifested disrupted nuclear architecture and abnormal epigenetic modifications. The absence of abnormal nuclear blabbing (FIG. 4C) and lamin A/C expression as well as the increased expression of lamin B1 and HDAC1 indicated that defects in the nucleus and epigenetics were reset in HGPS-iPSCs (FIG. 4D) and reprogramming rejuvenated the premature aging in HGPS cells.

### Example 5—Recapitulation of Premature Aging in HGPS-iPSCS Derived MSCS (HGPS-MSCS)

HGPS-iPSC's were further induced to generate MSCs using our aforementioned protocol. The differentiation of both WT-iPSCs and HGPS-iPSCs were performed in parallel. At passage 3, the MSCs differentiated from both WT-iPSCs and HGPS-iPSCs exhibited typical MSC surface markers with over 95% of cells positive for CD73, CD90, CD105 and CD44. Meanwhile, the MSCs negative markers were extremely low, with less than 0.1% of cells positive for CD11b, CD20, CD34, CD45 and HLA-DR (FIGS. 9A-9J). These MSCs were able to further differentiate into adipocytes, osteocytes and chondrocytes (FIG. 9K).

Further characterization of HGPS-MSCs by double staining of lamin B2 and progerin revealed the presence of progerin and nuclear abnormality. About half of the HGPS-MSCs (47.8±10.3%) exhibited detectable progerin expression. Significant higher percentage of cells manifested nuclear blabbing (78.1±12.7% in HGPS-MSCs Vs 21.1±6.8% in WT-MSCs) (FIG. 4E). HGPS-MSCs exhibited significantly reduced proliferation (57.3±5.8% in WT-MSCs Vs 7.9±1.6% in HGPS-MSCs) (FIG. 4F) and remarkably increased senescence (86.7±5.6% in HGPS-MSCs Vs 7.5±4.3% WT-MSCs) and increased DNA damage (FIG. 9L). In line with these observations and our previous findings (Liu et al. 2005), the DNA-damage checkpoint response in HGPS-MSCs was defective upon 10 Gy of γ-irradiation, as indicated by the delayed recruitment of 53BP1 (FIG. 10). As expected, HGPS-MSCs manifested senescence associated secretory phenotypes (SASP) with dramatic upregulation of IL1a, IL1b, IL6, IL8 and PAI (FIGS. 4H-4I). In addition, significant reduction in heterochromatin markers, such as HP1a, H3K9me3 and H3K27me3 were observed in HGPS-MSCs (FIG. 11). Collectively, these results demonstrated that HGPS-MSCs derived by our protocol recapitulate the phenotypes observed in HGPS cells and can serve as a cell model for laminopathy-based premature aging.

### Example 6—Correction of Pathogenic Mutation in HGPS Cells by a CRISPR/Cas9-based Double Selection System

As MSCs transplantation can ameliorate aging associated disorders and extend lifespan,⁴³ it is plausible MSCs will serve as a novel therapeutic strategy for HGPS in addition to other approaches like targeting lamin A post translational process, modulating progeria or prelamin A level, activation of autophagy and reprogramming⁴⁴⁻⁴⁶. To fulfil this purpose and avoid potential undesired immune response, we generated genetically rectified autologous MSCs. The homologous recombination (HR) strategy was adopted to correct HGPS mutation in iPSCs derived from the patient. Earlier HR methods mainly employ a single antibiotic resistant gene cassette to enrich positive cells, which requires an additional round of screening to remove DNA fragment. To accelerate this process, we designed a double selection donor vector containing antibiotic resistant gene and fluorescent protein mCherry gene cassette flanking with a 3.5 kb and 1.7 kb homologous arm, respectively (FIG. 5A). To correct HGPS-iPSC, the guide RNA out of gene body region was designed and a specificity-enhanced Cas9 was used to minimize the undesired side-effects caused by gene editing. After nucleofection and puromycin selection, colonies were collected and expanded to screen for HGPS-iPSC clones with LMNA gene correction by Sanger sequencing (FIG. 5B). The mutation-corrected HGPS-iPSCs clone (HGPS-CiPSCs) was further examined for its pluripotency. As shown in FIG. 5C, the HGPS-CiPSCs were positive for SSEA4, OCT3/4 and TRA1-60. Karyotyping assay confirmed the genome integrity in HGPS-CiPSCs (FIG. 5D). We then generated MSCs from HGPS-CiPSCs. As shown in FIGS. 5E-5I, surface markers confirmed HGPS-CiPSCs-derived MSCs met the criteria of MSCs (FIGS. 5E-5I). HGPS-CiPSCs-derived MSCs showed a significant downregulation of p16 and a dramatic decline in SASP-associated genes, compared to their counterpart HGPS-MSCs (FIG. 5J). To examine the fate of these MSCs in vivo, we labelled both HGPS-MSCs-derived and HGPS-CiPSC-derived MSCs with luciferase via lentiviral infection. Equal number (10⁶ cells) of HGPS-MSCs-derived and HGPS-CiPSC-derived MSCs were inoculated into immunocompromised mice at the middle parts of left and right tibialis anterior (TA) muscle, respectively. Luminescence was measured every other day. At day 7, accelerated decay of luminescence was observed in the left leg at the engrafted site (FIG. 5K), indicating HGPS-CiPSC derived MSCs have significant survival capability in vivo. These data collectively demonstrated rejuvenation of premature senescence after mutation correction.

### Example 7—Use of Generated MSC for Clinical Applications

MSCs hold a great promise for a broad-range of clinical applications and regenerative medicine. Concerns over the immune response and ethical issue on the allogenic MSCs have been in debate whereas the application of autologous MSCs is constrained by the limited number of primary MSCs available. A number of methods and protocols have been developed to derive MSCs from hPSCs.^{19, 20, 22, 23, 47, 48, 49, 50} However, these methods take relatively long time and are less efficient. The present invention provides a rapid yet robust method to generate multipotent MSCs from human pluripotent stem cells including ESCs and iPSCs. It has been reported that MSCs can be derived from pure NCCs by a two-stage induction strategy (Fukuta et al., 2014; PNAS, 2013/4). It involves either extended culture time or purification of NCCs by FACS sorting, making it difficult to obtain large scale MSCs within a short period of time. The subject methods initially can induce the PSCs to a state of neural ectoderm within about 5 to about 7 days, followed by MSC differentiation within as short as 4 days. Fully-differentiated MSCs from iPSCs or ESCs can be obtained within about 2 about 3 weeks, which is much shorter than current protocols through extended NCC induction that usually takes at least 5 weeks. The subject methods provide for the generation MSCs in a large scale without sorting, making it quite practical for clinical applications. Importantly, these PSCs-derived MSCs exhibited standard surface markers with about 90% similarity in transcriptomic profiles and comparable differentiation potential demonstrated by tri-lineage induction, when compared with bone marrow derived primary MSCs.

HGPS is an extremely rare autosomal dominant genetic disorder in which aging associated symptoms are manifested at early stage of life. HGPS provides a useful model for aging study to reveal the underlying molecular mechanism and therapeutic strategies for physiological aging and ageing related diseases. To extend the application of our method, we generated a cell model for HGPS by reprogramming the dermal fibroblasts from a HGPS patient into iPSCs, followed by MSCs induction. One concern for clinical application of PSCs-derived MSCs is the insertional mutations by foreign DNA. To minimize the potential detrimental effects of foreign DNA during the reprogramming, we employed minicircle vector to express Yamanaka factors to avoid insertional mutations in the genome. The HGPS-MSCs derived from the progeria patient manifested irregular nuclear morphology and blabbing, increased DNA damage, defective DNA damage repair, abnormal epigenetic modification, inflammatory cytokines activation and accelerated senescence. Therefore, HGPS-MSCs can serves as a cell model for accelerated aging in vitro for mechanistic study and senolytic screening. As reprogramming silenced the LMNA gene in HGPS-iPSCs which is devoid of senescence, it provides an unlimited source for the production of MSCs to serve as a cellular model for HGPS.

To further extend the potential clinical application of our method, we generated LMNA mutation-rectified MSCs from HGPS-iPSCs. As shown in FIGS. 5A-5J, LMNA mutation-rectified MSCs lost the production of progerin and inflammatory cytokines. Importantly, mutation-corrected MSCs were rejuvenated with a significantly improved survival when transplanted into immunocompromised mice. This provides a proof of principle in the application of our method by generating genetically rectified MSCs for autologous stem cell therapy in premature aging. The reprogramming process and MSCs induction were carried out with chemically-defined medium without animal components, making it complaint with potential clinical application.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Friedenstein A J, Chailakhjan R K, Lalykina K S. The development of fibroblast colonies in monolayer cultures of guinea-pig bone marrow and spleen cells. Cell Tissue Kinet. October 1970; 3 (4): 393-403. doi: 10.1111/j.1365-2184.1970.tb00347.x
2. Dominici M, Le Blanc K, Mueller I, Slaper-Cortenbach I, Marini F C, Krause D S, Deans R J, Keating A, Prockop D J, Horwitz E M. Minimal criteria for defining multipotent mesenchymal stromal cells. The International Society for Cellular Therapy position statement. Cytotherapy. August 2006; 8 (4): 315-317. doi: 10.1080/14653240600855905
3. Pittenger M F, Discher D E, Peault B M, Phinney D G, Hare J M, Caplan A I. Mesenchymal stem cell perspective: cell biology to clinical progress. NPJ Regen Med. 2019; 4:22. doi: 10.1038/s41536-019-0083-6
4. Bianco P, Robey P G, Simmons P J. Mesenchymal stem cells: revisiting history, concepts, and assays. Cell Stem Cell. Apr. 10, 2008; 2 (4): 313-9. doi: 10.1016/j.stem.2008.03.002
5. Singer N G, Caplan A I. Mesenchymal stem cells: mechanisms of inflammation. Annu Rev Pathol. 2011; 6:457-78. doi: 10.1146/annurev-pathol-011110-130230
6. Kyurkchiev D, Bochev I, Ivanova-Todorova E, Mourdjeva M, Oreshkova T, Belemezova K, Kyurkchiev S. Secretion of immunoregulatory cytokines by mesenchymal stem cells. World J Stem Cells. Nov. 26, 2014; 6 (5): 552-70. doi: 10.4252/wjsc.v6.15.552
7. Park C W, Kim K S, Bae S, Son H K, Myung P K, Hong H J, Kim H. Cytokine secretion profiling of human mesenchymal stem cells by antibody array. Int J Stem Cells. May 2009; 2 (1): 59-68. doi: 10.15283/ijsc.2009.2.1.59
8. Wang M, Yuan Q, Xie L. Mesenchymal Stem Cell-Based Immunomodulation: Properties and Clinical Application. Stem Cells Int. 2018; 2018:3057624. doi: 10.1155/2018/3057624
9. Squillaro T, Peluso G, Galderisi U. Clinical Trials With Mesenchymal Stem Cells: An Update. Cell Transplant. 2016; 25 (5): 829-48. doi: 10.3727/096368915X689622
10. Kozlowska U, Krawczenko A, Futoma K, Jurek T, Rorat M, Patrzalek D, Klimczak A. Similarities and differences between mesenchymal stem/progenitor cells derived from various human tissues. World J Stem Cells. Jun. 26, 2019; 11 (6): 347-374. doi: 10.4252/wjsc.v11.i6.347
11. Elahi K C, Klein G, Avci-Adali M, Sievert K D, MacNeil S, Aicher W K. Human Mesenchymal Stromal Cells from Different Sources Diverge in Their Expression of Cell Surface Proteins and Display Distinct Differentiation Patterns. Stem Cells Int. 2016; 2016:5646384. doi: 10.1155/2016/5646384
12. Mitxitorena I, Infante A, Gener B, Rodriguez C I. Suitability and limitations of mesenchymal stem cells to elucidate human bone illness. World J Stem Cells. Sep. 26, 2019; 11 (9): 578-593. doi: 10.4252/wjsc.v11.i9.578
13. Jiang T, Xu G, Wang Q, Yang L, Zheng L, Zhao J, Zhang X. In vitro expansion impaired the stemness of early passage mesenchymal stem cells for treatment of cartilage defects. Cell Death Dis. Jun. 1, 2017; 8 (6): e2851. doi: 10.1038/cddis.2017.215
14. Beane O S, Fonseca V C, Cooper L L, Koren G, Darling E M. Impact of aging on the regenerative properties of bone marrow-, muscle-, and adipose-derived mesenchymal stem/stromal cells. PLOS One. 2014; 9 (12): e115963. doi: 10.1371/journal.pone.0115963
15. Bernardo M E, Fibbe W E. Mesenchymal stromal cells: sensors and switchers of inflammation. Cell Stem Cell. Oct. 3, 2013; 13 (4): 392-402. doi: 10.1016/j.stem.2013.09.006
16. Griffin M D, Ryan A E, Alagesan S, Lohan P, Treacy O, Ritter T. Anti-donor immune responses elicited by allogeneic mesenchymal stem cells: what have we learned so far? Immunol Cell Biol. January 2013; 91 (1): 40-51. doi: 10.1038/icb.2012.67
17. Huang X P, Sun Z, Miyagi Y, McDonald Kinkaid H, Zhang L, Weisel R D, Li R K. Differentiation of allogeneic mesenchymal stem cells induces immunogenicity and limits their long-term benefits for myocardial repair. Circulation. Dec. 7, 2010; 122 (23): 2419-29. doi: 10.1161/CIRCULATIONAHA.110.955971
18. Kotobuki N, Katsube Y, Katou Y, Tadokoro M, Hirose M, Ohgushi H. In vivo survival and osteogenic differentiation of allogeneic rat bone marrow mesenchymal stem cells (MSCs). Cell Transplant. 2008; 17 (6): 705-12. doi: 10.3727/096368908786092793
19. Barberi T, Willis L M, Socci N D, Studer L. Derivation of multipotent mesenchymal precursors from human embryonic stem cells. PLOS Med. June 2005; 2 (6): e161. doi: 10.1371/journal.pmed.0020161
20. Lian Q, Lye E, Suan Yeo K, Khia Way Tan E, Salto-Tellez M, Liu™, Palanisamy N, El Oakley R M, Lee E H, Lim B, et al. Derivation of clinically compliant MSCs from CD105+, CD24-differentiated human ESCs. Stem Cells. February 2007; 25 (2): 425-36. doi: 10.1634/stemcells.2006-0420
21. Sanchez L, Gutierrez-Aranda I, Ligero G, Rubio R, Munoz-Lopez M, Garcia-Perez J L, Ramos V, Real P J, Bueno C, Rodriguez R, et al. Enrichment of human ESC-derived multipotent mesenchymal stem cells with immunosuppressive and anti-inflammatory properties capable to protect against experimental inflammatory bowel disease. Stem Cells. February 2011; 29 (2): 251-62. doi: 10.1002/stem.569
22. Zhao Q, Gregory C A, Lee R H, Reger R L, Qin L, Hai B, Park M S, Yoon N, Clough B, McNeill E, et al. MSCs derived from iPSCs with a modified protocol are tumor-tropic but have much less potential to promote tumors than bone marrow MSCs. Proc Natl Acad Sci USA. Jan. 13, 2015; 112 (2): 530-5. doi: 10.1073/pnas.1423008112
23. Wang X, Lazorchak A S, Song L, Li E, Zhang Z, Jiang B, Xu R H. Immune modulatory mesenchymal stem cells derived from human embryonic stem cells through a trophoblast-like stage. Stem Cells. February 2016; 34 (2): 380-91. doi: 10.1002/stem.2242
24. Leung A W, Murdoch B, Salem A F, Prasad M S, Gomez G A, Garcia-Castro M I. WNT/beta-catenin signaling mediates human neural crest induction via a pre-neural border intermediate. Development. Feb. 1, 2016; 143 (3): 398-410. doi: 10.1242/dev.130849
25. Fukuta M, Nakai Y, Kirino K, Nakagawa M, Sekiguchi K, Nagata S, Matsumoto Y, Yamamoto T, Umeda K, Heike T, et al. Derivation of mesenchymal stromal cells from pluripotent stem cells through a neural crest lineage using small molecule compounds with defined media. PLOS One. 2014; 9 (12): e112291. doi: 10.1371/journal.pone.0112291
26. Menendez L, Yatskievych T A, Antin P B, Dalton S. Wnt signaling and a Smad pathway blockade direct the differentiation of human pluripotent stem cells to multipotent neural crest cells. Proc Natl Acad Sci USA. Nov. 29, 2011; 108 (48): 19240-5. doi: 10.1073/pnas. 1113746108
27. Banito A, Rashid S T, Acosta J C, Li S, Pereira C F, Geti I, Pinho S, Silva J C, Azuara V, Walsh M, et al. Senescence impairs successful reprogramming to pluripotent stem cells. Research Support, Non-U.S. Gov't. Genes Dev. Sep. 15, 2009; 23 (18): 2134-9. doi: 10.1101/gad.1811609
28. Srinivasan A, Toh Y C. Human Pluripotent Stem Cell-Derived Neural Crest Cells for Tissue Regeneration and Disease Modeling. Front Mol Neurosci. 2019; 12:39. doi: 10.3389/fnmol.2019.00039
29. Liu Q, Spusta S C, Mi R, Lassiter R N, Stark M R, Hoke A, Rao M S, Zeng X. Human neural crest stem cells derived from human ESCs and induced pluripotent stem cells: induction, maintenance, and differentiation into functional schwann cells. Stem Cells Transl Med. April 2012; 1 (4): 266-78. doi: 10.5966/sctm.2011-0042
30. Huang M, Miller M L, McHenry L K, Zheng T, Zhen Q, Ilkhanizadeh S, Conklin B R, Bronner M E, Weiss W A. Generating trunk neural crest from human pluripotent stem cells. Sci Rep. Jan. 27, 2016; 6:19727. doi: 10.1038/srep19727
31. Jiang X, Gwye Y, Mckeown S J, Bronner-Fraser M, Lutzko C, Lawlor E R. Isolation and characterization of neural crest stem cells derived from in vitro-differentiated human embryonic stem cells. Stem Cells Dev. September 2009; 18 (7): 1059-70. doi: 10.1089/scd.2008.0362
32. Alvarez-Viejo M, Menendez-Menendez Y, Otero-Hernandez J. CD271 as a marker to identify mesenchymal stem cells from diverse sources before culture. World J Stem Cells. Mar. 26, 2015; 7 (2): 470-6. doi: 10.4252/wjsc.v7.12.470
33. Li W, Huang L, Zeng J, Lin W, Li K, Sun J, Huang W, Chen J, Wang G, Ke Q, et al. Characterization and transplantation of enteric neural crest cells from human induced pluripotent stem cells. Mol Psychiatry. March 2018; 23 (3): 499-508. doi: 10.1038/mp.2016.191
34. Lee G, Kim H, Elkabetz Y, Al Shamy G, Panagiotakos G, Barberi T, Tabar V, Studer L. Isolation and directed differentiation of neural crest stem cells derived from human embryonic stem cells. Nat Biotechnol. December 2007; 25 (12): 1468-75. doi: 10.1038/nbt1365
35. Gordon L B, Rothman F G, Lopez-Otin C, Misteli T. Progeria: a paradigm for translational medicine. Cell. Jan. 30, 2014; 156 (3): 400-7. doi: 10.1016/j.cell.2013.12.028
36. Scaffidi P, Misteli T. Lamin A-dependent nuclear defects in human aging. Science. May 19 2006; 312 (5776): 1059-63. doi: 10.1126/science. 1127168
37. Kubben N, Zhang W, Wang L, Voss T C, Yang J, Qu J, Liu G H, Misteli T. Repression of the Antioxidant NRF2 Pathway in Premature Aging. Cell. Jun. 2 2016; 165 (6): 1361-1374. doi: 10.1016/j.cell.2016.05.017
38. Osmanagic-Myers S, Kiss A, Manakanatas C, Hamza O, Sedlmayer F, Szabo P L, Fischer I, Fichtinger P, Podesser B K, Eriksson M, et al. Endothelial progerin expression causes cardiovascular pathology through an impaired mechanoresponse. J Clin Invest. Feb. 1, 2019; 129 (2): 531-545. doi: 10.1172/JCI121297
39. Hamczyk M R, Villa-Bellosta R, Gonzalo P, Andres-Manzano M J, Nogales P, Bentzon J F, Lopez-Otin C, Andres V. Vascular Smooth Muscle-Specific Progerin Expression Accelerates Atherosclerosis and Death in a Mouse Model of Hutchinson-Gilford Progeria Syndrome. Circulation. Jul. 17 2018; 138 (3): 266-282. doi: 10.1161/CIRCULATIONAHA.117.030856
40. Zhang J Q, Lian Q Z, Zhu G L, Zhou F, Sui L, Tan C, Mutalif R A, Navasankari R, Zhang Y L, Tse H F, et al. A Human iPSC Model of Hutchinson Gilford Progeria Reveals Vascular Smooth Muscle and Mesenchymal Stem Cell Defects. Cell Stem Cell. Jan. 7, 2011; 8 (1): 31-45. doi: 10.1016/j.stem.2010.12.002
41. Jung H J, Coffinier C, Choe Y, Beigneux A P, Davies B S, Yang S H, Barnes R H, 2nd, Hong J, Sun T, Pleasure S J, et al. Regulation of prelamin A but not lamin C by miR-9, a brain-specific microRNA. Proc Natl Acad Sci USA. Feb. 14 2012; 109 (7): E423-31. doi: 10.1073/pnas. 1111780109
42. Nissan X, Blondel S, Navarro C, Maury Y, Denis C, Girard M, Martinat C, De Sandre-Giovannoli A, Levy N, Peschanski M. Unique preservation of neural cells in Hutchinson-Gilford progeria syndrome is due to the expression of the neural-specific miR-9 microRNA. Cell Rep. Jul. 26 2012; 2 (1): 1-9. doi: 10.1016/j.celrep.2012.05.015
43. Shen J, Tsai Y T, Dimarco N M, Long M A, Sun X, Tang L. Transplantation of mesenchymal stem cells from young donors delays aging in mice. Sci Rep. 2011; 1:67. doi: 10.1038/srep00067
44. Harhouri K, Frankel D, Bartoli C, Roll P, De Sandre-Giovannoli A, Levy N. An overview of treatment strategies for Hutchinson-Gilford Progeria syndrome. Nucleus. Jan. 1, 2018; 9 (1): 246-257. doi: 10.1080/19491034.2018.1460045
45. Guilbert S M, Cardoso D, Levy N, Muchir A, Nissan X. Hutchinson-Gilford progeria syndrome: Rejuvenating old drugs to fight accelerated ageing. Methods. Apr. 9, 2020; doi: 10.1016/j.ymeth.2020.04.005
46. Ocampo A, Reddy P, Martinez-Redondo P, Platero-Luengo A, Hatanaka F, Hishida T, Li M, Lam D, Kurita M, Beyret E, et al. In Vivo Amelioration of Age-Associated Hallmarks by Partial Reprogramming. Cell. Dec. 15 2016; 167 (7): 1719-1733 e12. doi: 10.1016/j.cell.2016.11.052
47. Trivedi P, Hematti P. Derivation and immunological characterization of mesenchymal stromal cells from human embryonic stem cells. Exp Hematol. March 2008; 36 (3): 350-9. doi: 10.1016/j.exphem.2007.10.007
48. Xu C, Jiang J, Sottile V, McWhir J, Lebkowski J, Carpenter M K. Immortalized fibroblast-like cells derived from human embryonic stem cells support undifferentiated cell growth. Stem Cells. 2004; 22 (6): 972-80. doi: 10.1634/stemcells.22-6-972
49. Mahmood A, Harkness L, Schroder H D, Abdallah B M, Kassem M. Enhanced differentiation of human embryonic stem cells to mesenchymal progenitors by inhibition of TGF-beta/activin/nodal signaling using SB-431542. J Bone Miner Res. June 2010; 25 (6): 1216-33. doi: 10.1002/jbmr.34
50. Karlsson C, Emanuelsson K, Wessberg F, Kajic K, Axell M Z, Eriksson P S, Lindahl A, Hyllner J, Strehl R. Human embryonic stem cell-derived mesenchymal progenitors—potential in regenerative medicine. Stem Cell Res. July 2009; 3 (1): 39-50. doi: 10.1016/j.scr.2009.05.002
51. Diecke S, Lu J, Lee J, Termglinchan V, Kooreman N G, Burridge P W, Ebert A D, Churko J M, Sharma A, Kay M A, et al. Novel codon-optimized mini-intronic plasmid for efficient, inexpensive, and xeno-free induction of pluripotency. Sci Rep. Jan. 28 2015; 5:8081. 10 doi: 10.1038/srep08081
52. Li T, Jin W, He Y, Zhou Z. Generation of PRMT6 homozygous knockout human embryonic stem cell lines. Stem Cell Res. Dec. 28 2020; 50:102136. doi: 10.1016/j.scr.2020.102136

1. A method of producing Mesenchymal stem cells (MSCs) from mammalian pluripotent stem cells, the method comprising:
culturing the mammalian pluripotent stem cells for a first time period in a first medium comprising a cell basal medium, a cell culture supplement, a non-essential amino acids solution, sodium pyruvate, insulin, transferrin, selenium, L-ascorbic acid, an antibiotic solution, a GSK-3 inhibitor, and an inhibitor of activin receptor-like kinase receptors.

2. The method of claim 1, further comprising:
culturing the MSCs for a second time period in a second medium containing a basal medium, FBS, a cell culture supplement, bFGF, TGFB1, and an antibiotic solution.

3. The method of claim 1, wherein the first time period is about 1 to about 8 days, about 2 to about 6 days, or about 4 days.

4. The method of claim 2, wherein the second time period is about 1 to about 14 days, about 2 to about 12 days, or about 4 to about 10 days.

5. The method of claim 1, wherein the basal medium is DMEM: F12 basal medium.

6. The method of claim 1, wherein the cell culture supplement is an L-alanyl-L-glutamine dipeptide.

7. The method of claim 1, wherein the non-essential amino acid solution comprises at least one of glycine, L-alanine, L-asparagine, L-aspartic acid, L-glutamic acid, L-proline, and L-serine.

8. The method of claim 1, wherein the L-ascorbic acid is at a concentration of about 1 μg/mL to about 100 μg/mL or about 50 μg/mL.

9. The method of claim 1, wherein the antibiotic solution comprises penicillin and streptomycin.

10. The method of claim 1, wherein the GSK-3 inhibitor is CHIR-99021 at a concentration of about 1 μM to about 100 UM or about 10 μM.

11. The method of claim 1, wherein the inhibitor of activin receptor-like kinase receptors is SB-431542 at a concentration of about 1 μM to about 100 μM or about 10 μM.

12. The method of claim 2, wherein the basal medium is alpha-MEM basal medium.

13. The method of claim 2, wherein the FBS is at a concentration of about 1% to about 15% or about 10%.

14. The method of claim 2, wherein the cell culture supplement is an L-alanyl-L-glutamine dipeptide.

15. The method of claim 2, wherein the bFGF is at a concentration of about 0.1 ng/ml to about 10 ng/ml or about 1 ng/mL.

16. The method of claim 2, wherein the TGFβ1 is at a concentration of about 0.1 ng/ml to about 10 ng/mL or about 1 ng/mL.

17. The method of claim 2, wherein the antibiotic solution comprises penicillin and streptomycin.

18. The method of claim 1, wherein the pluripotent stem cells are embryonic stem cells.

19. The method of claim 1, wherein the pluripotent stem cells are human pluripotent stem cells.


SEQUENCE LISTING (72 sequences)

SEQ ID NO: 1 - DNA, 19 bp, synthetic construct
  source 1..19: mol_type = other DNA
ggcagaagggcaagagaag

SEQ ID NO: 2 - DNA, 20 bp, synthetic construct
  source 1..20: mol_type = other DNA
ctcccgccatctgttgttag

SEQ ID NO: 3 - DNA, 21 bp, synthetic construct
  source 1..21: mol_type = other DNA
gtgcctggcaccgccatcaat

SEQ ID NO: 4 - DNA, 23 bp, synthetic construct
  source 1..23: mol_type = other DNA
cgagaagccgctccacatacagt

SEQ ID NO: 5 - DNA, 22 bp, synthetic construct
  source 1..22: mol_type = other DNA
gacggctgtggctggaagttcg

SEQ ID NO: 6 - DNA, 21 bp, synthetic construct
  source 1..21: mol_type = other DNA
ccgcttggcctcgtcgatgaa

SEQ ID NO: 7 - DNA, 21 bp, synthetic construct
  source 1..21: mol_type = other DNA
gacaggtgcctctgcggccaa

SEQ ID NO: 8 - DNA, 21 bp, synthetic construct
  source 1..21: mol_type = other DNA
cgtacgccttggagccgtaca

SEQ ID NO: 9 - DNA, 23 bp, synthetic construct
  source 1..23: mol_type = other DNA
ctagcgagggcctatttcccatg

SEQ ID NO: 10 - DNA, 20 bp, synthetic construct
  source 1..20: mol_type = other DNA
tgtctcgaggtcgagaattc

SEQ ID NO: 11 - DNA, 20 bp, synthetic construct
  source 1..20: mol_type = other DNA
agctacagcatgatgcagga

SEQ ID NO: 12 - DNA, 20 bp, synthetic construct
  source 1..20: mol_type = other DNA
ggtcatggagttgtactgca

SEQ ID NO: 13 - DNA, 20 bp, synthetic construct
  source 1..20: mol_type = other DNA
tgaacctcagctacaaacag

SEQ ID NO: 14 - DNA, 20 bp, synthetic construct
  source 1..20: mol_type = other DNA
tggtggtaggaagagtaaag

SEQ ID NO: 15 - DNA, 20 bp, synthetic construct
  source 1..20: mol_type = other DNA
tcgctgagctgaaacaaatg

SEQ ID NO: 16 - DNA, 20 bp, synthetic construct
  source 1..20: mol_type = other DNA
cccttcttgaaggtttacac

SEQ ID NO: 17 - DNA, 20 bp, synthetic construct
  source 1..20: mol_type = other DNA
tctcaaggcacacctgcgaa

SEQ ID NO: 18 - skipped

SEQ ID NO: 19 - DNA, 20 bp, synthetic construct
  source 1..20: mol_type = other DNA
actctgaggaggaacaagaa

SEQ ID NO: 20 - DNA, 19 bp, synthetic construct
  source 1..19: mol_type = other DNA
tggagacgtggcacctctt

SEQ ID NO: 21 - DNA, 23 bp, synthetic construct
  source 1..23: mol_type = other DNA
aagctacacacaggacttgacag

SEQ ID NO: 22 - DNA, 20 bp, synthetic construct
  source 1..20: mol_type = other DNA
agttcggacagctgggcttt

SEQ ID NO: 23 - DNA, 20 bp, synthetic construct
  source 1..20: mol_type = other DNA
tgtgcaccaacatctacaag

SEQ ID NO: 24 - DNA, 20 bp, synthetic construct
  source 1..20: mol_type = other DNA
gcgttcttggctttcaggat

SEQ ID NO: 25 - DNA, 20 bp, synthetic construct
  source 1..20: mol_type = other DNA
aaatgtttgtgttgcggtca

SEQ ID NO: 26 - DNA, 20 bp, synthetic construct
  source 1..20: mol_type = other DNA
tctggcacaggtgtcttcag

SEQ ID NO: 27 - DNA, 20 bp, synthetic construct
  source 1..20: mol_type = other DNA
gtaacccgttgaaccccatt

SEQ ID NO: 28 - DNA, 20 bp, synthetic construct
  source 1..20: mol_type = other DNA
ccatccaatcggtagtagcg

SEQ ID NO: 29 - DNA, 20 bp, synthetic construct
  source 1..20: mol_type = other DNA
caaagttgtcatggatgacc

SEQ ID NO: 30 - DNA, 18 bp, synthetic construct
  source 1..18: mol_type = other DNA
ccatggagaaggctgggg

SEQ ID NO: 31 - DNA, 22 bp, synthetic construct
  source 1..22: mol_type = other DNA
gctgctcctcaactatgctaag

SEQ ID NO: 32 - DNA, 25 bp, synthetic construct
  source 1..25: mol_type = other DNA
gaattcagtgctgcttcatattctc

SEQ ID NO: 33 - DNA, 22 bp, synthetic construct
  source 1..22: mol_type = other DNA
agttggacgaggtcaacaagag

SEQ ID NO: 34 - DNA, 20 bp, synthetic construct
  source 1..20: mol_type = other DNA
ggactccaggtccttcacac

SEQ ID NO: 35 - DNA, 21 bp, synthetic construct
  source 1..21: mol_type = other DNA
ctcagtgactgtggttgagga

SEQ ID NO: 36 - DNA, 17 bp, synthetic construct
  source 1..17: mol_type = other DNA
agtgcaggctcggcctc

SEQ ID NO: 37 - DNA, 21 bp, synthetic construct
  source 1..21: mol_type = other DNA
ctcagtgactgtggttgagga

SEQ ID NO: 38 - DNA, 18 bp, synthetic construct
  source 1..18: mol_type = other DNA
ttctgggggctctgggct

SEQ ID NO: 39 - DNA, 21 bp, synthetic construct
  source 1..21: mol_type = other DNA
aatgatcgcttggcggtctac

SEQ ID NO: 40 - DNA, 21 bp, synthetic construct
  source 1..21: mol_type = other DNA
cacctcttcagactcggtgat

SEQ ID NO: 41 - DNA, 20 bp, synthetic construct
  source 1..20: mol_type = other DNA
ctgcttccaggaaactccac

SEQ ID NO: 42 - DNA, 21 bp, synthetic construct
  source 1..21: mol_type = other DNA
gccagggcagaaaagcagaag

SEQ ID NO: 43 - DNA, 22 bp, synthetic construct
  source 1..22: mol_type = other DNA
ttgggatgtgtagagtttgaga

SEQ ID NO: 44 - DNA, 25 bp, synthetic construct
  source 1..25: mol_type = other DNA
taaaccaaaacaatccttctactcc

SEQ ID NO: 45 - DNA, 20 bp, synthetic construct
  source 1..20: mol_type = other DNA
attgacctacagtgcccagc

SEQ ID NO: 46 - DNA, 20 bp, synthetic construct
  source 1..20: mol_type = other DNA
atgctttggaaattgacgga

SEQ ID NO: 47 - DNA, 18 bp, synthetic construct
  source 1..18: mol_type = other DNA
cggaagcaggtttggaga

SEQ ID NO: 48 - DNA, 17 bp, synthetic construct
  source 1..17: mol_type = other DNA
cggagacgctccggact

SEQ ID NO: 49 - DNA, 19 bp, synthetic construct
  source 1..19: mol_type = other DNA
caggctttgcagcactcac

SEQ ID NO: 50 - DNA, 20 bp, synthetic construct
  source 1..20: mol_type = other DNA
ctgctgctgttgctgcttct

SEQ ID NO: 51 - DNA, 20 bp, synthetic construct
  source 1..20: mol_type = other DNA
gcatgttcagctgggaaatc

SEQ ID NO: 52 - DNA, 20 bp, synthetic construct
  source 1..20: mol_type = other DNA
atgctgtgtttggccttctt

SEQ ID NO: 53 - DNA, 20 bp, synthetic construct
  source 1..20: mol_type = other DNA
ctttcttgtgctgcatacgg

SEQ ID NO: 54 - DNA, 18 bp, synthetic construct
  source 1..18: mol_type = other DNA
agctcagcaagacgctgg

SEQ ID NO: 55 - DNA, 19 bp, synthetic construct
  source 1..19: mol_type = other DNA
cacgtgcatgtgcttcttg

SEQ ID NO: 56 - DNA, 20 bp, synthetic construct
  source 1..20: mol_type = other DNA
gcgctccgagaatttaaaga

SEQ ID NO: 57 - DNA, 19 bp, synthetic construct
  source 1..19: mol_type = other DNA
agcgaacgcacatcaagac

SEQ ID NO: 58 - DNA, 20 bp, synthetic construct
  source 1..20: mol_type = other DNA
ctgtaggcgatctgttgggg

SEQ ID NO: 59 - DNA, 19 bp, synthetic construct
  source 1..19: mol_type = other DNA
cggtcggaggccgatccag

SEQ ID NO: 60 - DNA, 22 bp, synthetic construct
  source 1..22: mol_type = other DNA
gcgccgtggagcagcagcagct

SEQ ID NO: 61 - DNA, 21 bp, synthetic construct
  source 1..21: mol_type = other DNA
cctgtcactgtcttgtaccct

SEQ ID NO: 62 - DNA, 22 bp, synthetic construct
  source 1..22: mol_type = other DNA
gcgtttggagtggtagaaatct

SEQ ID NO: 63 - DNA, 23 bp, synthetic construct
  source 1..23: mol_type = other DNA
gtgctgctgaaggagatgcctga

SEQ ID NO: 64 - DNA, 22 bp, synthetic construct
  source 1..22: mol_type = other DNA
cccctgccaagcacacccagta

SEQ ID NO: 65 - DNA, 20 bp, synthetic construct
  source 1..20: mol_type = other DNA
tgcacgctccgggactcaca

SEQ ID NO: 66 - DNA, 25 bp, synthetic construct
  source 1..25: mol_type = other DNA
catggagaacaccacttgttgctcc

SEQ ID NO: 67 - DNA, 20 bp, synthetic construct
  source 1..20: mol_type = other DNA
ccaggagcccagctatgaac

SEQ ID NO: 68 - DNA, 19 bp, synthetic construct
  source 1..19: mol_type = other DNA
cccagggagaaggcaactg

SEQ ID NO: 69 - DNA, 20 bp, synthetic construct
  source 1..20: mol_type = other DNA
gagtggaccacactgcgcca

SEQ ID NO: 70 - DNA, 22 bp, synthetic construct
  source 1..22: mol_type = other DNA
tccacaaccctctgcacccagt

SEQ ID NO: 71 - DNA, 21 bp, synthetic construct
  source 1..21: mol_type = other DNA
cctggcctcagacttcggggt

SEQ ID NO: 72 - DNA, 23 bp, synthetic construct
  source 1..23: mol_type = other DNA
ggggccatgcccttgtcatcaat